(19) United States
(12) Patent Application Publication
(10) Pub. No.: US 2017/0319698 A1
(43) Pub. Date: Nov. 9, 2017

(54) GASTRORETENTIVE GEL FORMULATIONS

(71) Applicant: Jagotec AG, Muttenz (CH)

(72) Inventors: Guy VERGNAULT, Kembs (FR); Ubaldo CONTE, Busto Arsizio (IT); Lauretta MAGGI, Pavia (IT)

(21) Appl. No.: 15/523,145

(22) PCT Filed: Oct. 26, 2015
(86) PCT No.: PCT/EP2015/002117

§ 371 Date: Apr. 28, 2017

(30) Foreign Application Priority Data
Oct. 29, 2014 (GB) 1419257.9

(51) Int. Cl. A61K 47/14; A61K 47/38; A61K 9/06; A61K 47/44; A61K 9/00

(52) U.S. Cl. CPC A61K 47/14; A61K 47/44; A61K 9/0053; A61K 9/06; A61K 47/38

(57) ABSTRACT

The present invention provides a gastric retentive gel composition comprising: (a) a hydrophobic or amphiphilic liquid gelled with an organogelator; (b) an active agent; and (c) a hard wax or wax-like additive, for controlled delivery of the active agent to or through the upper gastrointestinal tract, in particular to or through the stomach. The gel composition forms a stable, floating and coherent raft in the gastric environment, and is not directly expelled from the stomach as a result of gastric emptying. The active agent is released from the composition in a controlled manner for absorption or local action.

The present disclosure relates to gastroretentive gel formulations, which may be useful for controlled delivery of an active agent to or through the upper gastrointestinal tract, in particular to or through the stomach.

### BACKGROUND OF THE DISCLOSURE

Oral administration is a highly favoured route for drug delivery. An orally administered formulation is taken in the mouth and swallowed, for local action in the gastrointestinal (GI) tract or absorption into the bloodstream. Oral administration is simple, convenient, pain free and does not require any specialized equipment or the involvement of a healthcare professional, thereby promoting better patient compliance and lower cost.

A drug formulation entering the stomach via the oral route will typically remain there only for a short and unpredictable length of time, owing to gastric emptying which occurs as part of the normal digestive process. The short gastric residence time limits the efficacy and bioavailability of many types of orally administered drugs, including, in particular, drugs which are intended for local action in the stomach, and drugs which are preferentially absorbed in the upper GI tract or which are unstable or have low solubility at neutral or alkaline pH in the environment of the lower GI tract. Drugs of this type may be advantageously delivered by way of a gastroretentive formulation or drug delivery system which is adapted to remain in the stomach for an extended period of time, notwithstanding gastric emptying.

Gastroretentive systems also permit better control of drug delivery. In the case of orally administered drugs having a slow absorption profile, a gastroretentive delivery system can enable adequate gastric retention time for absorption of the drug. Conversely, for orally administered drugs which are very rapidly absorbed, a gastroretentive delivery system is capable of achieving controlled release and delivery of a large quantity of drug over a period of time.

Various gastroretentive drug delivery systems have been proposed in the art. High density systems, designed to sink in the stomach for retention in the stomach wall below the pyloric sphincter, have been studied in ruminant mammals with some encouraging results, but the effectiveness of such systems in humans has not been proved, and no products for human use have been marketed. Expandable systems including a device adapted to increase in size in the stomach environment, thereby precluding expulsion into the duodenum, have also been developed. These systems however are complex to manufacture, and, in use, leave behind a device or carrier in the stomach after the drug has been released, leading to a risk of obstruction, intestinal adhesion and gastropathy. Muco/bioadhesive systems have also been studied, with mixed success, owing to the rapid turnover of mucus in the GI tract.

Floating drug delivery systems show greater promise as a means for achieving gastroretention of pharmaceutical formulations. Floating systems available in the art include effervescent systems, which are caused to float by the production and release of gas by effervescent components, and hydrodynamically balanced systems comprising hydrophilic matrices which swell and expand in the gastric environment to attain a density lower than the density of the contents of the stomach. Hydrophilic gels and matrices are however inherently unstable in the gastric environment, and show a tendency to disaggregate, resulting in uncontrolled release of the drug. The function and performance of aqueous gels is also notably affected by the nature of the stomach contents, and is therefore unpredictable and inefficient.

### SUMMARY OF THE DISCLOSURE

The present disclosure accordingly seeks to provide an improved gastroretentive floating system for delivery of an active agent to or through the upper gastrointestinal tract of a human or animal patient. In particular, the present disclosure seeks to provide a system which enables controlled and sustained release of an active agent into the upper GI tract. The present disclosure seeks to provide a system which is easily swallowable and acceptable to patients.

In accordance with one aspect of the present disclosure therefore there is provided a gastric retentive gel composition comprising: (a) a hydrophobic or amphiphilic liquid gelled with an organogelator; (b) an active agent; and (c) a hard wax or wax-like additive. An organogelator is a gelling agent which is capable of forming a linked network, typically a cross-linked network, through a liquid organic phase, in this case a hydrophobic or amphiphilic liquid, to yield a stable gel (component (a) of the composition). Suitably, the gel may be an organogel, a lipogel or an oleogel. Oleogels are known in the art for use in the food industry and in the cosmetic and pharmaceutical industries for topical application—see, for example, Ruiz Martinez et al, Il Farmaco 58 (2003) 1289-1294; Dassanayake et al, Current Opinion in Colloid & Interface Science 16 (2011) 432-439; Zetzl et al, Food Funct 2012, 3, 327-337 etc. However, the use of oleogels in floating gastric retentive drug delivery systems has not previously been described.

The gel composition of the present disclosure further includes a hard wax or wax-like additive (component (c) of the composition). The hard wax or wax-like additive may be dispersed, suspended or solubilised in the gel of component (a). The present inventors have surprisingly found that the inclusion of a hard wax or wax-like additive in a gel composition according to the present disclosure helps to maintain the integrity of the composition in an aqueous environment, by resisting fluid ingress. This improves the stability of the composition in the gastric environment and enables it to hold together as a raft whilst the active agent is released. This also enables a larger quantity of active agent to be incorporated into the gel composition without causing the composition to sink. Furthermore, as demonstrated hereinbelow, the inclusion of a hard wax or wax-like additive enables better control of the release of the active agent from the composition, with a more stable and slower release profile. The homogeneity of the gel composition is also improved, with more even dispersion of the active agent and other substances or materials.

Gel compositions including an active ingredient for oral administration are described in EP 0356325. EP 0356325 is not however concerned with gastric retentive formulations. Furthermore, EP 0356325 does not contemplate the inclusion of a hard wax or wax-like additive in its compositions.

The gel composition of the present disclosure is gastric retentive, which means that, on oral administration to a patient, the composition is retained in the stomach for a period of time exceeding normal gastric retention time of conventional dosage forms. The composition is not directly expelled from the stomach into the duodenum as a result of normal gastric emptying. This is because the gel composition holds together and floats as a raft on the contents of the stomach. The buoyancy of the composition in gastric fluid is affected by a range of different factors, including its density, bioadhesivity, hydrophilicity and the presence of additional ingredients. These factors are selected in accordance with the present disclosure to ensure that the gel composition of the present disclosure will hold together and float as a raft on the contents of the stomach, and thus be gastrically retained.

Advantageously, the composition of the present disclosure may be suitable for administration by way of a sachet, such as a squeezable sachet; or a bottle; or a tube; or a dosing syringe; or a bottle with a dosing pump. Thus, a further aspect of the present disclosure comprehends a dosage form which is a sachet, bottle, tube, dosing syringe, or a bottle with a dosing pump comprising the composition of the present disclosure. Suitably, the dosage form may be adapted to enable convenient delivery and administration of a predetermined quantity of the composition, representing a dose of said active agent. Thus, the dosage form may be a single dose sachet containing a predetermined quantity of the composition; or a bottle, tube or syringe that is configured to dispense a predetermined quantity of the composition representing a dose of the active agent.

In a further aspect, the present disclosure provides a method for administering an active agent to or through the upper gastrointestinal tract, in particular the stomach, of a human or animal patient, comprising orally administering to the patient a gel composition according to the present disclosure which comprises said active agent. Preferably, the patient is in a non-fasting state, which means that the stomach of the patient is not empty. Suitably, the patient may have eaten or may be instructed to eat food no more than 2 hours, preferably no more than 1 hour, before oral administration of the composition, or may eat or be instructed to eat food just before or in conjunction with oral administration of the composition.

In a further aspect, the present disclosure provides a gel composition comprising an active agent, in accordance with the present disclosure, for use in gastric delivery of the active agent to a human or animal patient. The present disclosure further provides a gel composition comprising an active agent, in accordance with the present disclosure, for use in administering said active agent to or through the upper gastrointestinal tract, in particular the stomach, of a human or animal patient. The present disclosure further provides a gel composition in accordance with the present disclosure for use in oral administration to a human or animal patient. Preferably, the patient is in a non-fasting state, which means that the stomach of the patient is not empty. Suitably, the patient may have eaten or may be instructed to eat food no more than 2 hours, preferably no more than 1 hour, before oral administration of the composition, or may eat or be instructed to eat food just before or in conjunction with oral administration of the composition.

In yet a further aspect, the present disclosure provides the use of a composition in accordance with the present disclosure in the manufacture of a medicament for gastric delivery of said active agent.

### DETAILED DESCRIPTION OF THE DISCLOSURE

The present disclosure provides a gastric retentive gel composition comprising: (a) a hydrophobic or amphiphilic liquid gelled with an organogelator; (b) an active agent; and (c) a hard wax or wax-like additive.

Said hydrophobic or amphiphilic liquid may suitably be a physiologically acceptable liquid. Said hydrophobic or amphiphilic liquid may advantageously have an HLB (hydrophilic-lipophilic balance) value of 10 or less, preferably 8 or less, more preferably 6 or less. Said hydrophobic or amphiphilic liquid may suitably be in liquid form at or near physiological temperature.

Suitably, component (a) of the composition of the present disclosure may comprise an organogel, lipogel or oleogel. Component (a) of the composition of the present disclosure preferably comprises a stable and uniform gel, formed by a polymeric network in a non-aqueous hydrophobic or amphiphilic liquid. Methods for the production of such gels are known in the art. For example, the hydrophobic or amphiphilic liquid component may be combined with the organogelator, and heated with constant stirring to enable adequate dispersion of the organogelator through the hydrophobic or amphiphilic liquid. This mixture is then allowed to cool and set as a gel. Suitably, the active agent and/or the hard wax or wax-like additive may subsequently be dispersed, solubilised or suspended in the gel. This may be readily achieved by mixing.

The gel composition of the present disclosure is a gastric retentive composition and is adapted to float on the contents of the stomach. Preferably, the gel composition of the present disclosure may form a continuous, substantially continuous, partially fragmented or fragmented floating raft on the top of the contents of the stomach. The buoyancy of the composition is affected by various different factors, including its density. To facilitate satisfactory floating, the gel composition of the present disclosure may preferably have a density which is less than the density of gastric fluid (approximately 1.004-1.1 g/ml). The gel composition may have a density which is less than about 1.1 g/ml, preferably less than about 1.05 g/ml, or less than about 1.04 g/ml, or less than about 1.03 g/ml, or less than about 1.02 g/ml, or less than about 1.01 g/ml, or less than about 1.005 g/ml, or less than about 1.004 g/ml, or less than about 1 g/ml. Preferably, the gel composition of the present disclosure may be capable of remaining afloat in vitro in dissolution medium, such as distilled water. The skilled person may readily adjust the formulation of the composition, in particular the relative quantities of components (a), (b) and (c) and the nature and quantity of the organogelator in component (a), to achieve an appropriate density to ensure floating.

The buoyancy of the gel composition and its ability to act as a raft is also affected by other physical features, including cohesivity, consistency, hydrophilicity, viscosity and bioadhesivity. As exemplified hereinbelow, the inclusion of a hard wax (component c) has been found to improve the cohesivity of the composition and help it to hold together as a raft in an aqueous medium (such as a dissolution medium in vitro, or the gastric environment in vivo) without falling apart and breaking into multiple small flakes. The hydrophobic nature of the hard wax may also help to reduce the ingress of water into the composition from an aqueous environment, and hence to resist sinking.

The gel composition of the present disclosure is preferably non-aqueous or substantially non-aqueous. “Non-aqueous or substantially non-aqueous” in this context means that the composition is formulated with no added water or with less than 5%, or less than 4%, or less than 3%, or less than 2%, or less than 1%, or less than 0.5% added water. Preferably, each of components (a), (b) and/or (c) of the composition may be non-aqueous or substantially non-aqueous. In contrast to hydrogel formulations in the art, which show a tendency to disaggregate owing to the ingress of water, the gel composition of the present disclosure has superior integrity in the gastric environment.

The gel composition of the present disclosure may preferably have a semi-solid consistency and may preferably be soft and uniform in texture. A soft texture and semi-solid consistency should promote easy swallowing and therefore make the composition more acceptable to patients. Furthermore, a soft semi-solid gel is unlikely to cause discomfort or unpleasant sensations whilst it is retained in the stomach. The components of the composition are preferably biocompatible and biodegradable.

The gel composition of the present disclosure includes a hard wax or wax-like additive. The inclusion of the hard wax or wax-like additive surprisingly enhances the properties and characteristics of the gel composition. A wax is an organic compound, typically comprising long alkyl chains, which is insoluble or has very low solubility in water, and is malleable at or near room temperature. Waxes may be natural, semi-synthetic or synthetic. Specific examples of waxes include animal waxes, vegetable waxes, mineral waxes, petroleum waxes and synthetic waxes. A wax-like material is a material which has similar characteristics and physical properties to a wax, whilst having a different chemical structure.

Suitably, the hard wax or wax-like additive may have a melting point above room temperature; preferably above about 30° C., or above about 32° C., or above about 35° C., or above about 37° C., or above about 40° C., or above about 45° C., or above about 50° C., or above about 55° C. or above about 60° C. or above about 65° C., or above about 70° C. The hard wax or wax-like additive may be crystalline or microcrystalline and may be insoluble or poorly soluble in water.

The hard wax or wax-like additive may comprise a single hard wax or wax-like material or a mixture of hard wax or wax-like materials. The or at least one hard wax or wax-like material in the composition may be natural, semi-synthetic or synthetic. The or at least one hard wax or wax-like material in the composition may be of animal, mineral or vegetal origin.

In some embodiments, the or at least one hard wax or wax-like material in the composition may be selected from triglycerides, mono- and diglycerides, natural or synthetic waxes, fat or wax alcohols, fatty acids, esters of fatty alcohols and fatty acids, or fatty acid amides. Suitably, the or at least one hard wax or wax-like material in the composition may be selected from mono-, di- or tri-glycerides of C₅₋₂₁ or C₈₋₂₁ or C₈₋₁₈ fatty acids. Optionally, the or at least one hard wax or wax-like material in the composition may be selected from mono-, di- or tri-glycerides of saturated, unbranched and/or unsubstituted fatty acids. In particular, the or at least one hard wax or wax-like material in the composition may be selected from mono-, di- or tri-glycerides of palmitic acid, stearic acid, or behenic acid.

In some embodiments, the or at least one hard wax or wax-like material in the composition may comprise a hardened fat or oil, such as a fat or oil hardened by partial hydrogenation. Said hardened fat or oil may be a hardened vegetable oil such as hardened castor oil, peanut oil, soybean oil, colza oil, rapeseed oil, cottonseed oil, soybean oil, sunflower oil, palm oil, palm kernel oil, linseed oil, almond oil, corn oil, olive oil, sesame oil, cocoa butter or coconut fat, or shea butter.

In some embodiments, the or at least one hard wax or wax-like material in the composition may be selected from Suppocire® (a semi-synthetic glyceride base comprising saturated C8-18 triglyceride fatty acids) such as Suppocire® NA, Suppocire® AM or Suppocire® AML (contains lecithin); Witepsol® (a mixture of triglycerides, diglycerides and monoglycerides) from H, W or S range; Gelucire® (a mixture of glycerides and esters of polyethylene glycol), preferably Gelucire® having a low hydrophilic-lipophilic balance (HLB), for example an HLB below 4, such as Gelucire® 43/01; Geleol™ (mono- and diglycerides, glycerol monostearate); Cutina® HR (hydrogenated castor oil); Cutina® GMS (a glyceryl stearate); Softisan 54 (hydrogenated palm oil); Syncrowax® HRC (triglyceride of behenic acid); Compritol® 888 ATOS (glyceryl behenate) or Compritol® HD5 (Behenoyl Polyoxyl-8 glycerides); Precirol® (glyceryl distearate); Imwitor® (a glyceryl stearate) such as Imwitor® 900 and Imwitor® 191; or Dynasan® (114—trimyristate; 116—tripalmitate; 118—tristearate), or equivalents of any of these. These materials are all readily commercially available, for example from Gattefosse, Croda or Huls, and are familiar to those skilled in the art.

In some embodiments, the or at least one hard wax or wax-like material in the composition may comprise a C₁₂₋₅₀ fatty alcohol. The fatty alcohol may preferably be saturated. Optionally, the fatty acid may be obtained from a natural fat, oil or wax. The fatty alcohol may optionally be selected from myristyl alcohol, 1-pentadecanol, cetyl alcohol, 1-heptadecanol, stearyl alcohol, 1-nona-decanol, arachidyl alcohol, 1-heneicosanol, behenyl alcohol, brassidyl alcohol, lignoceryl alcohol, ceryl alcohol or myricyl alcohol; or may comprise a fatty alcohol cut obtained in the reduction of naturally occurring fats and oils such as, for example, bovine tallow, peanut oil, colza oil, cottonseed oil, soybean oil, sunflower oil, palm kernel oil, linseed oil, castor oil, corn oil, rapeseed oil, sesame oil, cocoa butter and coconut oil. Alternatively, the fatty alcohol may be of synthetic origin; and may optionally be selected from linear, even-numbered fatty alcohols from Ziegler's synthesis (Alfols), partly branched alcohols from the oxosynthesis (Dobanols), C₁₄₋₂₂ fatty alcohols marketed for example by Cognis Deutschland GmbH under the name of Lanette® 16 (C16 alcohol), Lanette® 14 (C₁₄alcohol), Lanette® 0 (C_{16/18} alcohol) or Lanette® 22 (C_{18/22} alcohol).

In some embodiments, the or at least one hard wax or wax-like material in the composition may be selected from C14-40 fatty acids or mixtures thereof; such as myristic, pentadecanoic, palmitic, margaric, stearic, nonadecanoic, arachic, behenic, lignoceric, cerotic, melissic, erucic and elaeostearic acid or substituted fatty acids such as 12-hydroxystearic acid, or the amides or monoethanolamides of the fatty acids.

In some embodiments, the or at least one hard wax or wax-like material in the composition may be selected from natural vegetable waxes, such as candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, or animal waxes such as, for example, beeswax, shellac wax, spermaceti, wool wax or uropygial fat.

In some embodiments, the or at least one hard wax or wax-like material in the composition may be selected from natural mineral waxes such as ceresine and ozocerite, and/or petrochemical waxes, such as petrolatum, paraffin waxes or microwaxes, and/or chemically modified waxes, more particularly the hard waxes such as montan ester waxes, sasol waxes or hydrogenated jojoba waxes.

In some embodiments, the or at least one hard wax or wax-like material in the composition may be selected from the group of wax esters of saturated and/or unsaturated, branched and/or unbranched alkanecarboxylic acids and saturated and/or unsaturated, branched and/or unbranched alcohols; from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols; or from the group of lactides of long-chain hydroxycarboxylic acids. Optionally, the wax ester may be selected from C₁₆₋₄₀ alkyl stearates, C₂₀₋₄₀ alkyl stearates (for example Kesterwachs® K82H), C₂₀₋₄₀ dialkyl esters of dimer acids, C₁₈₋₃₈ alkyl hydroxystearoyl stearates or C₂₀₋₄₀ alkyl erucates.

In some embodiments, the or at least one hard wax or wax-like material in the composition may be selected from C₃₀₋₅₀ alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, or cetearyl behenate.

The gel composition of the present disclosure comprises a gel formed by gelation of a hydrophobic or amphiphilic liquid with an organogelator (component (a) of the composition). The hard wax or wax-like additive of component (c) may be included in the gel composition in a quantity ranging from about 5-200% by weight of component (a)—excluding the active agent (component (b)) and any additives or additional ingredients in the composition. The hard wax or wax-like additive may be included in an amount ranging from about 5-100% or 100-200% by weight of component (a). The hard wax or wax-like additive may be provided in an amount ranging from about 5-10%, or 10-20%, or 20-30%, or 30-40%, or 40-50%, or 50-60%, or 60-70%, or 70-80%, or 80-90%, or 90-100%, or 100-110%, or 110-120%, or 120-130%, or 130-140%, or 140-150%, or 150-160%, or 160-170%, or 170-180%, or 180-190%, or 190-200% by weight of component (a).

The organogelator is a component which is capable of forming a gel with the hydrophobic or amphiphilic liquid. Suitably, the gel should be capable of holding together as a raft and floating in the gastric environment. Various suitable organogelators are known in the art, including low molecular weight organogelators and polymeric organogelators. Preferably, in a composition of the present disclosure, the organogelator may comprise a polymeric component. The polymeric component may comprise a cellulose polymer such as a methylcellulose, ethylcellulose or hydroxypropyl cellulose polymer; preferably a cellulose polymer or ethylcellulose polymer having a viscosity grade of at least about 3 cP or at least about 5 cP or at least about 10 cP or at least about 20 cP, and/or up to about 80 cP or up to about 100 cP. Suitably, the polymeric component may comprise a cellulose polymer or ethylcellulose polymer having a viscosity grade of about 50 cP or less.

Ethylcellulose polymers are widely available in the art and are known as organogelators for the preparation of oleogels. The ethylcellulose polymer may therefore comprise Ethocel™ 4, Ethocel™ 10, Ethocel™ 14, Ethocel™ 20, Ethocel™ 45, Ethocel™ 50, Ethocel™ 70, or Ethocel™ 100, or equivalents of any of these (such as Aqualon® N50). These polymers are commercially available, for example from Dow Chemical Company.

In other embodiments, the polymeric component may comprise polyethylene, polyethyleneoxides such as Polyox™ WSRN-80, propyleneoxides, gums such as xanthan gum, guar gum or locust bean gum, polyacrylic acids or polyacrylic-methacrylic acids such as Eudragit® (ammonio methacrylate copolymer), shellac resin, polyvinyl acids, carboxyvinyl polymers such as carbomers, or block copolymer ethyleneoxide-co-propyleneoxides including poloxamers, such as Lutrol® F127.

The organogelator is gelled with the hydrophobic or amphiphilic liquid to form a gel (component (a)). Preferably, the w/w ratio of organogelator to hydrophobic or amphiphilic liquid in the gel is up to about 15:85 (15% organogelator), or up to about 10:90 (10% organogelator), or up to about 9:91 (9% organogelator), or up to about 8:92 (8% organogelator), or up to about 7:93 (7% organogelator), or up to about 6:94 (6% organogelator), or up to about 5:95 (5% organogelator), or between about 1:99-5:95 (1-5% organogelator), or between about 2:98-5:95 (2-5% organogelator), or between about 3:97-5:95 (3-5% organogelator), or about 4:96 (4% organogelator) or less. It will be appreciated that these percentages refer to the relative quantities of the organogelator and the hydrophobic or amphiphilic liquid in component (a), excluding the hard wax or wax-like additive, the active agent and any other additives in the gel composition.

In one particularly preferred embodiment, component (a) of the composition of the present disclosure comprises a hydrophobic or amphiphilic liquid gelled with an ethylcellulose polymer, and the w/w ratio of the ethylcellulose polymer to the hydrophobic or amphiphilic liquid is about 4:96 (4% ethylcellulose polymer). Preferably, the ethylcellulose polymer may have a viscosity grade of about 50 cP.

Suitably, in a composition according to the present disclosure, the hydrophobic or amphiphilic liquid may comprise a fat, oil or butter. In particular, the hydrophobic or amphiphilic liquid may comprise an oil, a fractionated oil or oil fraction including a glyceride or fatty acid fraction, a purified oil, an oil derivative, or a modified oil. A modified oil may be an oil which has been modified physically or chemically so as to alter the oil from its natural state. For example, a modified oil may be an oil which has been depleted or enriched with certain fractions, such as fatty acid and/or glyceride fractions, and/or to alter the ratio of different constituents, including monoglycerides, diglycerides and triglycerides. A modified oil may additionally or alternatively be an oil which has been chemically modified through the removal or addition of chemical groups, for example through the grafting of propylene glycol groups. The oil must be non-toxic or edible and may for example be a vegetable oil, such as corn oil, olive oil, palm oil, sunflower oil, soybean oil, coconut oil, safflower oil, peanut oil, cottonseed oil, or rapeseed oil. The hydrophobic or amphiphilic liquid may comprise a glyceride such as a mono-, di- or tri-glyceride. The hydrophobic or amphiphilic liquid may be a commercially available product, such as Capryol™ such as Capryol™ 90 (propyleneglycol monocaprate), Labrafac™ such as Labrafac™ PG (propyleneglycol dicaprylocaprate), Labrafil® such as Labrafil® 1944 CS (oleoyl macrogol 6 glycerides), Lauroglycol™ such as Lauroglycol™ 90 (propyleneglycol monolaurate), Maisine™ such as Maisine™ 35-1 (glyceryl monolinoleate), Peceol™ (glycerol mono oleate), Plurol® Oleique (polyglyceryl 3 dioleate), a medium chain triglyceride (MCT) such as Miglyol® 810 or 812, or equivalents of any of these. These are all commercially available, for example from Gattefosse.

The active agent may comprise a pharmaceutical drug, a nutritional supplement, a marker such as a diagnostic marker, an imaging agent, or the like. Suitably, the active agent may be suitable for administration to or through the upper part of the gastrointestinal tract, in particular to or through the stomach. Thus, the composition may be a composition for gastric delivery of the active agent.

The active agent may for example be intended or administered for local action in the stomach, or may be intended or administered for absorption into the bloodstream. In particular, the active agent may comprise an agent which is preferentially absorbed through the upper part of the gastrointestinal tract or which shows better solubility in the upper part of the gastrointestinal tract, or an agent which is only slowly absorbed through the gastrointestinal tract, or an agent which shows low solubility at low or neutral pH, or an agent which is less enzymatically metabolised when absorbed through the proximal small intestine. Such agents are particularly suited to administration by way of a gastroretentive formulation. Preferably, the active agent may be suitable for gastric delivery. Suitably, therefore, the active agent should not for example cause gastric injury or show instability in the low pH of the stomach environment.

Suitable active agents may, in some embodiments, be for the prevention, mitigation, and/or treatment of diseases, conditions, and/or symptoms thereof in a patient. Examples of such diseases and conditions may include, but are not limited to, arthritis, rheumatoid arthritis, juvenile rheumatoid arthritis, psoriatic arthritis, osteoarthritis, gouty arthritis, refractory rheumatoid arthritis, chronic non-rheumatoid arthritis, osteoporosis/bone resorption, osteophorosis, ulcerative colitis, skin diseases, psoriasis, acne vulgaris, rosacea, dermatitis, contact dermatitis, eczema, delayed-type hypersensitivity in skin disorders, type I diabetes, type II diabetes, Alzheimer's disease, inflammatory disorders, immunodeficiency, inflammatory bowel disease, irritable bowel syndrome, Crohn's disease, diarrhea disease, antibiotic associated diarrhea, pediatric diarrhea, chronic constipation, heartburn, appendicitis, autoimmune disorders, multiple sclerosis, muscle degeneration, coeliac disease, diabetes mellitus, organ transplantation, bacterial infections, viral infections, fungal infections, periodontal disease, urogenital disease, sexually transmitted disease, HIV infection, HIV replication, HIV associated diarrhea, surgical associated trauma, surgical-induced metastatic disease, nausea, weight loss, weight gain, anorexia, bulimia, fever control, cachexia, wound healing, ulcers, gut barrier function, allergies, Hay Fever, allergic rhinitis, anaphylaxis, asthma, respiratory disorders, lung diseases, pulmonary fibrosis, chronic obstructive pulmonary disease, circulatory disorders, anemia, disorders of the blood coagulation system, renal disease, disorders of the central nervous system, hepatic disease, ischemia, nutritional disorders, endocrine disorders, epidermal disorders, multiple myeloma, uveititis, acute and chronic myelogenous leukemia, anti-clotting, coronary heart disease, vasculitis, ischemic heart disease, atherosclerosis, strokes, peripheral arterial disease, ischemic-induced cell damage, high blood cholesterol levels, high-density lipoprotein (HDL) levels, high blood pressure, pancreatic [beta] cell destruction, rheumatoid spondylitis, adult respiratory distress syndrome (ARDS), bone resorption diseases, ischemia reperfusion injury, brain trauma, cerebral malaria, sepsis, septic shock, toxic shock syndrome, blood infection, fever, myalgias due to infection, HIV-1, HIV-2, HIV-3, immune system disorders, cytomegalovirus, colds, influenza, adenovirus, the herpes viruses (including HSV-1, HSV-2), herpes zoster infection, herpes simplex/cold sores, infections, disorders associated with C-reactive protein, myositis, lupus, Celiac disease, prostatitis, tumor, sexual dysfunction, inflammatory disease, thyroid diseases, pregnancy, headaches, acute pain, rashes, addiction, addiction to habit forming drugs, addiction to smoking, upper respiratory tract infection, neurodegenerative disease, dyslexia, dyspraxia, autism, Asperger's disease, mild cognitive impairment, poor concentration, attention deficit disorder (ADD), attention deficit hyperactive disorder (ADHD), depression, mood swings, bipolar disorders, cancer, leukemia, acute and chronic myelogenous leukemia, colon cancer, prostate cancer, kidney cancer, liver cancer, breast cancer, lung cancer, melanoma, brain cancer, cervical cancer, Hodgkin's lymphoma, non-Hodgkin's lymphoma, ovarian cancer, testicular cancer, thyroid cancer, uterine cancer, urinary tract infection, nervous system infection, and the like.

Nonlimiting examples of agents suitable for use in conjunction with the present disclosure may include, but are not limited to, active pharmaceuticals, prodrugs of active pharmaceuticals, active biologicals, anthelmintics, antibiotics, antifungals, antitoxins, antigens, therapeutics, preventive therapeutics, analgesics, nutritional supplements, imaging agents, fluid stabilizers, flavorants, or any combination thereof.

Examples of suitable active pharmaceuticals and prodrugs of active pharmaceuticals for use in the present disclosure may include, but are not limited to, 16-alpha fluoroestradiol, 16-alpha-gitoxin, 16-epiestriol, 17-alpha dihydroequilenin, 17-alpha estradiol, 17-beta estradiol, 17-hydroxy progesterone, 1-alpha-hydroxyvitamin D2, 1-dodecpyrrolidinone, 20-epi-1,25 dihydroxyvitamin D3, 22-oxacalcitriol, 2CW, 2′-nor-cGMP, 3-isobutyl GABA, 5-ethynyluracil, 6-FUDCA, 7-methoxytacrine, abamectin, abanoquil, abcizimab, abecarnil, abiraterone, ablukast, ablukast sodium, acadesine, acamprosate, acarbose, acebutolol, acecamide hydrochloride, aceclidine, aceclofenac, acedapsone, aceglutamide aluminum, acemannan, acetaminophen, acetazolamide, acetohexamide, acetohydroxamic acid, acetomepregenol, acetophenazine maleate, acetosulfone sodium, acetylcholine chloride, acetylcysteine, acetyl-L-carnitine, acetylmethadol, acifran, acipimox, acitemate, acitretin, acivicin, aclarubicin, aclatonium, acodazole hydrochloride, aconiazide, acrisorcin, acrivastine, acronine, actisomide, actodigin, acyclovir, acylfulvene, adafenoxate, adalimumab, adapalene, adapalene, adatanserin, adatanserin hydrochloride, adecypenol, adecypenol, adefovir, adelmidrol, ademetionine, adenosine, adinazolam, adipheinine hydrochloride, adiposin, adozelesin, adrafinil, adrenalone, airbutamine, alacepril, alamecin, alanine, alaproclate, alaptide, albendazole, albolabrin, albuterol, albutoin, alclofenae, alclometasone dipropionate, aluminum chlorhydroxyallantoinate, aldecalmycin, aldesleukin, aldioxa, alendronate sodium, alendronic acid, alentemol, alentemol hydrobromide, aletamine hydrochloride, aleuronium chloride, alexidine, alfacalcidol, alfentanil hydrochloride, alfuzosin, algestone acetonide, alglucerase, aliflurane, alinastine, alipamide, allantoin, allobarbital, allopurinol, a tachy-kinins (TK) antagonist, alonimid, alosetron, alosetron hydrochloride, alovudine, alpertine, alpha amylase, alpha idosone, alpidem, alprazolam, alprenolol hydrochloride, alprenoxime hydrochloride, alprostadil, alrestatin sodium, altanserin tartrate, alteplase, althiazide, altretamine, altromycin B, alverinc citrate, alvircept sudotox, amadinone acetate, amantadine hydrochloride, ambamustine, ambomycin, ambruticin, ambuphylline, ambuside, amcinafal, amcinonide, amdinocillin, amdinocillin pivoxil, amedalin hydrochloride, amelometasone, ameltolide, amesergide, ametantrone acetate, amezinium metilsulfate, amfebutamone, amfenac sodium, amflutizole, amicycline, amidephrine mesylate, amidox, amifloxacin, amifostine, amikacin, amiloride hydrochloride, aminacrine hydrochloride, aminobenzoate potassium, aminobenzoate sodium, aminocaproic acid, aminoglutethimide, aminohippurate sodium, aminolevulinic acid, aminophylline, aminorex, aminosalicylate sodium, aminosalicylic acid, amiodarone, amiprilose hydrochloride, amiquinsin hydrochloride, amisulpride, amitraz, amitriptyline hydrochloride, amlexanox, amlodipine, amobarbital sodium, amodiaquine, amodiaquine hydrochloride, amorolfine, amoxapine, amoxicillin, amphecloral, amphetamine sulfate, amphomycin, amphotericin B, ampicillin, ampiroxicam, ampyzine sulfate, amquinate, amrinone, aminone, amrubicin, amsacrine, amythiamicin, anagestone acetate, anagrelide, anakinra, ananain, anaritide, anaritide acetate, anastrozole, anazolene sodium, ancrod, andrographolide, androstenedione, angiogenesis inhibitors, angiotensin amide, anidoxime, anileridine, anilopam hydrochloride, aniracetam, anirolac, anisotropine methylbromide, anistreplase, anitrazafen, anordrin, antagonist D, antagonist G, antarelix, antazoline phosphate, anthelmycin, anthralin, anthramycin, antiandrogen, antihemophilic factor, acedapsone, felbamate, antiestrogen, antineoplaston, antipyrine, antisense oligonucleotides, apadoline, apafant, apalcillin sodium, apaxifylline, apazone, aphidicolin glycinate, apixifylline, apomorphine hydrochloride, apraclonidine, apraclonidine hydrochloride, apramycin, aprindine, aprindine hydrochloride, aprosulate sodium, aprotinin, aptazapine maleate, aptiganel, apurinic acid, apurinic acid, aranidipine, aranotin, arbaprostil, arbekicin, 1-methyl-2-((phenylthio)methyl)-3-carbethoxy-4-((dimethylamino)methyl)-5-hydroxy-6-bromindole, arbutamine hydrochloride, arclofenin, ardeparin sodium, (2R,4R)-1-[(2S)-5-(diaminomethylideneamino)-2-[[(3R)-3-methyl-1,2,3,4-tetrahydroquinolin-8-yl] sulfonylamino] pentanoyl]-4-methyl-piperidine-2-carboxylic acid, arginine, argipressin tannate, arildone, aripiprazol, arotinolol, arpinocid, arteflene, artilide fumarate, asimadoline, aspalatone, asparaginase, aspartic acid, aspartocin, asperfuran, aspirin, aspoxicillin, asprelin, astemizole, astromicin sulfate, asulacrine, atamestane, atenolol, atevirdine, atipamezole, atiprosin maleate, atolide, atorvastatin, atosiban, atovaquone, atpenin B, atracurium besylate, atrimustine, atrinositol, atropine, auranofin, aureobasidin A, aurothioglucose, avilamycin, avoparcin, avridine, nizatidine, axinastatin 1, axinastatin 2, axinastatin 3, azabon, azacitidinie, azaclorzine hydrochloride, azaconazole, azadirachtine, azalanstat dihydrochloride, azaloxan fumarate, azanator maleate, azanidazole, azaperone, azaribine, azaserine, azasetron, azatadine maleate, azathioprine, azathioprine sodium, azatoxin, azatyrosine, azelaic acid, azelastine, azelnidipine, azepindole, azetepa, azimilide, azithromycin, azlocillin, azolimine, azosemide, azotomycin, aztreonam, azumolene sodium, bacampicillin hydrochloride, baccatin III, bacitracin, baclofen, bacoside A, bacoside B, bactobolamine, balanol, balazipone, balhimycin, balofloxacin, balsalazide, bambermycins, bambuterol, bamethan sulfate, bamifylline hydrochloride, bamidazole, baohuoside 1, barmastine, barnidipine, basifungin, batanopride hydrochloride, batebulast, batelapine maleate, batimastat, beauvericin, becanthone hydrochloride, becaplermin, becliconazole, beclomethasone dipropionate, befloxatone, beinserazide, belfosdil, belladonna, beloxamide, bemesetron, bemitradine, bemoradan, benapryzine hydrochloride, benazepril hydrochloride, benazeprilat, bendacalol mesylate, bendazac, bendroflumethiazide, benflumetol, benidipine, benorterone, benoxaprofen, benoxaprofen, benoxinate hydrochloride, benperidol, bentazepam, bentiromide, benurestat, benzbromarone, benzethonium chloride, benzetimide hydrochloride, benzilonium bromide, benzindopyrine hydrochloride, benzisoxazole, benzocaine, benzochlorins, benzoctamine hydrochloride, benzodepa, benzoidazoxan, benzonatate, benzoyl peroxide, benzoylpas calcium, benzoylstaurosporine, benzquinamide, benzthiazide, benztropine, benztropine mesylate, benzydamine hydrochloride, benzylpenicilloyl polylysine, bepridil, bepridil hydrochloride, beractant, beraprost, berefrine, berlafenone, bertosamil, berythromycin, besipirdine, beta-alethine, betaclamycin B, betamethasone, betamipron, betaxolol, betaxolol hydrochloride, bethanechol chloride, bethanidine sulfate, betulinic acid, bevacizumab, bevantolol, bevantolol hydrochloride, bezafibrate, bFGF inhibitor, bialamicol hydrochloride, biapenem, bicalutamide, bicifadine hydrochloride, biclodil hydrochloride, bidisomide, bifemelane, bifonazole, bimakalim, bimithil, bindarit, biniramycin, binospirone, bioxalomycin alpha2, bipenamol hydrochloride, biperiden, biphenamine hydrochloride, biriperone, bisantrene, bisaramil, bisaziridinylspermine, bis-benzimidazole A, bis-benzimidazole B, bisnafide, bisobrin lactate, bisoprolol, bispyrithione magsulfex, bistramide D, bistramide K, bistratene A, bithionolate sodium, bitolterol besylate, bivalirudin, bizelesin, bleomycin sulfate, bolandiol dipropionate, bolasterone, boldenone undecylenate, boldine, bolenol, bolmantalate, bopindolol, bosentan, boxidine, brefeldin, breflate, brequinar sodium, bretazenil, bretylium bosylate, brifentanil hydrochloride, brimonidine, brinolase, brocresine, brocrinat, brofoxine, bromadoline maleate, bromazepam, bromchlorenone, bromelains, bromfenac, brominidione, bromocriptine, bromodiphenhydramine hydrochloride, bromoxamide, bromperidol, bromperidol decanoate, brompheniramine baleate, broperamole, bropirimine, brotizolam, bucamide maleate, bucindolol, buclizine hydrochloride, bucromarone, budesonide, budipine, budotitane, buformin, bumetamide, bunaprolast, bunazosin, bunolol hydrochloride, bupicomide, bupivacaine hydrochloride, buprenorphine hydrochloride, bupropion hydrochloride, buramate, buserelin acetate, buspirone hydrochloride, busulfan, butabarbital, butacetin, butaclamol hydrochloride, butalbital, butamben, butamirate citrate, butaperazine, butaprost, butedronate tetrasodium, butenafine, buterizine, buthionine sulfoximine, butikacin, butilfenin, butirosin sulfate, butixirate, butixocort propionate, butoconazole nitrate, butonate, butopamine, butoprozine hydrochloride, butorphanol, butoxamine hydrochloride, butriptyline hydrochloride, cactinomycin, cadexomer iodine, caffeine, calanolide A, calcifediol, calcipotriene, calcipotriol, calcitonin, calcitriol, calcium undecylenate, calphostin C, calusterone, cambendazole, camonagrel, camptothecin derivatives, canarypox IL-2, candesartan, candicidin, candoxatril, candoxatrilat, caniglibose, canrenoate potassium, canrenone, capecitabine, capobenate sodium, capobenic acid, capreomycin sulfate, capromab, capsaicin, captopril, capuride, caracemide, carbachol, carbadox, carbamazepine, carbamide peroxide, carbantel lauryl sulfate, carbaspirin calcium, carbazeran, carbazomycin C, carbenicillin potassium, carbenoxolone sodium, carbetimer, carbetocin, carbidopa, carbidopa-levodopa, carbinoxamine maleate, carbiphene hydrochloride, carbocloral, carbocysteine, carbol-fuchsin, carboplatin, carboprost, carbovir, carboxamide-amino-triazole, carboxyamidotriazole, carboxymethylated beta-1,3-glucan, carbuterol hydrochloride, CaRest M3, carfentanil citrate, carisoprodol, carmantadine, carmustine, CARN 700, camidazole, caroxazone, carperitide, carphenazine maleate, carprofen, carsatrin succinate, cartazolate, carteolol, carteolol hydrochloride, cartilage derived inhibitor, carubicin hydrochloride, carumonam sodium, carvedilol, carvotroline, carvotroline hydrochloride, carzelesin, casein kinase inhibitors (ICOS), castanospermine, caurumonam, cebaracetam, cecropin B, cedefingol, cefaclor, cefadroxil, cefamandole, cefaparole, cefatrizine, cefazaflur sodium, cefazolin, cefbuperazone, cefcapene pivoxil, cefdaloxime pentexil tosilate, cefdinir, cefditoren pivoxil, cefepime, cefetamet, cefetecol, cefixime, cefluprenam, cefinenoxime hydrochloride, cefinetazole, cefminlox, cefodizime, cefonicid sodium, cefoperazone sodium, ceforamide, cefoselis, cefotaxime sodium, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftibuten, ceftizoxime sodium, ceftriaxone, cefuroxime, celastrol, celikalim, celiprolol, cepacidiine A, cephacetrile sodium, cephalexin, cephaloglycin, cephaloridine, cephalothin sodium, cephapirin sodium, cephradine, cericlamine, cerivastatin, ceronapril, certoparin sodium, ceruletide, cetaben sodium, cetalkonium chloride, cetamolol hydrochloride, cetiedil, cetirizine, cetophenicol, cetraxate hydrochloride, cetrorelix, cetuximab, cetylpyridinium chloride, chenodiol, chlophedianol hydrochloride, chloral betaine, chlorambucil, chloramphenicol, chlordantoin, chlordiazepoxide, chlorhexidine gluconate, chlorins, chlormadinone acetate, chloroorienticin A, chloroprocaine hydrochloride, chloropropamide, chloroquine, chloroquinoxaline sulfonamide, chlorothiazide, chlorotrianisene, chloroxine, chloroxylenol, chlorphenesin carbamate, chlorpheniramine maleate, chlorpromazine, chlorpropamide, chlorprothixene, chlortetracycline bisulfate, chlorthalidone, chlorzoxazone, cholestyramine resin, chromonar hydrochloride, cibenzoline, cicaprost, ciclafrine hydrochloride, ciclazindol, ciclesonide, cicletanine, ciclopirox, cicloprofen, cicloprolol, cidofovir, cidoxepin hydrochloride, cifenline, ciglitazone, ciladopa hydrochloride, cilansetron, cilastatin sodium, cilazapril, cilnidipine, cilobamine mesylate, cilobradine, cilofungin, cilostazol, cimaterol, cimetidine, cimetropium bromide, cinalukast, cinanserin hydrochloride, cinepazet maleate, cinflumide, cingestol, cinitapride, cinnamedrine, cinnarizine, cinolazepam, cinoxacin, cinperene, cinromide, cintazone, cintriamide, cioteronel, cipamfylline, ciprefadol succinate, ciprocinonide, ciprofibrate, ciprofloxacin, ciprostene, ciramadol, cirolemycin, cisapride, cisatracurium besilate, cisconazole, cisplatin, cis-porphyrin, cistinexine, citalopram, citenamide, citicoline, citreamicin alpha, cladribine, clamoxyquin hydrochloride, clarithromycin, clausenamide, clavulanate potassium, clazolam, clazolimine, clebopride, clemastine, Clentiazem maleate, clidinium bromide, clinafloxacin, clindamycin, clioquinol, clioxamide, cliprofen, clobazam, clobetasol propionate, clobetasone butyrate, clocortolone acetate, clodanolene, clodazon hydrochloride, clodronic acid, clof azimine, clofibrate, clofilium phosphate, clogestone acetate, clomacran phosphate, clomegestone acetate, clometherone, clomethiazole, clomifene analogues, clominorex, clomiphene, clomipramine hydrochloride, clonazepam, clonidine, clonitrate, clonixeril, clonixin, clopamide, clopenthixol, cloperidone hydrochloride, clopidogrel, clopimozide, clopipazan mesylate, clopirac, cloprednol, cloprostenol sodium, clorazepate dipotassium, clorethate, clorexolone, cloroperone hydrochloride, clorprenaline hydrochloride, clorsulon, clortermine hydrochloride, closantel, closiramine aceturate, clothiapine, clothixamide maleate cloticasone propionate, clotrimazole, cloxacillin benzathine, cloxyquin, clozapine, cocaine, coccidioidin, codeine, codoxime, colchicine, colestimide, colestipol hydrochloride, colestolone, colforsin, colfosceril palmitate, colistimethate sodium, colistin sulfate, collismycin A, collismycin B, colterol mesylate, combretastatin A4, combretastatin analogue, complestatin, conagenin, conorphone hydrochloride, contignasterol, contortrostatin, cormethasone acetate, corticorelin ovine triflutate, corticotropin, cortisone acetate, cortivazol, cortodoxone, cosalane, costatolide, cosyntropin, cotinine, warfarin, coumermycin, crambescidin 816, crilvastatin, crisnatol, cromitrile sodium, cromolyn sodium, crotamiton, cryptophycin 8, cucumariosid, cuprimyxin, curacin A, curdlan sulfate, zinc hyaluran, cyclacillin, cyclazocine, cyclazosin, cyclic HPMPC, cyclindole, cycliramine maleate, cyclizine, cyclobendazole, cyclobenzaprine, cyclobut A, cyclobut G, cyclocapron, cycloguanil pamoate, cycloheximide, cyclopentanthraquinones, cyclopenthiazide, cyclopentolate hydrochloride, cyclophenazine hydrochloride, cyclophosphamide, cycloplatam, cyclopropane, cycloserine, cyclosin, cyclosporine, cyclothialidine, cyclothiazide, cyclothiazomycin, cyheptamide, cypemycin, cypenamine hydrochloride, cyprazepam, cyproheptadine hydrochloride, cyprolidol hydrochloride, cyproterone, cyproximide, cysteamine, cysteine hydrochloride, cystine, cytarabine, cytarabine hydrochloride, cytarabine ocfosfate, cytochalasin B, cytolytic factor, cytostatin, dacarbazine, dacliximab, dactimicin, dactinomycin, daidzein, daledalin tosylate, dalfopristin, dalteparin sodium, daltroban, dalvastatin, danaparoid, danazol, dantrolene, daphlnodorin A, dapiprazole, dapitant, dapoxetine hydrochloride, dapsone, daptomycin, darglitazone sodium, darifenacin, darlucin A, darodipine, darsidomine, darusentan, daunorubicin hydrochloride, dazadrol maleate, dazepinil hydrochloride, dazmegrel, dazopride fumarate, dazoxiben hydrochloride, debrisoquin sulfate, decitabine, deferiprone, deflazacort, dehydrocholic acid, dehydrodidemnin B, dehydroepiandrosterone, delapril, delapril hydrochloride, delavirdine mesylate, delequamine, delfaprazine, delmadinone acetate, delmopinol, delphinidin, demecarium bromide, demeclocycline, demecycline, demoxepam, denofungin, deoxypyridinoline, 2-propylpentanoic acid, deprodone, deprostil, depsidomycin, deramciclane, dermatan sulfate, desciclovir, descinolone acetonide, desflurane, desipramine hydrochloride, desirudin, deslanoside, deslorelin, desmopressin, desogestrel, desonide, desoximetasone, desoxoamiodarone, desoxycorticosterone acetate, detajmium bitartrate, deterenol hydrochloride, detirelix acetate, devazepide, dexamethasone, dexamisole, dexbrompheniramine maleate, dexchlorpheniramine maleate, dexclamol hydrochloride, dexetimide, dexfenfluramine hydrochloride, dexifosfamide, deximafen, dexivacaine, dexketoprofen, dexloxiglumide, dexmedetomidine, dexormaplatin, dexoxadrol hydrochloride, dexpanthenol, dexpemedolac, dexpropranolol hydrochloride, dexrazoxane, dexsotalol, dextrin 2-sulphate, dextroamphetamine, dextromethorphan, dextrorphan hydrochloride, dextrothyroxine sodium, dexverapamil, dezaguanine, dezinamide, dezocine, diacetolol hydrochloride, diamocaine cyclamate, diapamide, diatrizoate meglumine, diatrizoic acid, diaveridine, diazepam, diaziquone, diazoxide, dibenzepin hydrochloride, dibenzothiophene, dibucaine, dichliorvos, dichloralphenazone, dichlorphenamide, dicirenone, diclofenac sodium, dicloxacillin, dicranin, dicumarol, dicyclomine hydrochloride, didanosine, didemnin B, didox, dienestrol, dienogest, diethylcarbamazine citrate, diethylhomospermine, diethylnorspermine, diethylpropion hydrochloride, diethylstilbestrol, difenoximide hydrochloride, difenoxin, diflorasone diacetate, difloxacin hydrochloride, difluanine hydrochloride, diflucortolone, diflumidone sodium, diflunisal, difluprednate, diftalone, digitalis, digitoxin, digoxin, dihexyverine hydrochloride, dihydrexidine, dihydro-5-azacytidine, dihydrocodeine bitartrate, dihydroergotamine mesylate, hihydroestosterone, dihydrostreptomycin sulfate, dihydrotachysterol, dihydrotaxol, phenytoin, dilevalol hydrochloride, diltiazem hydrochloride, dimefadane, dimefline hydrochloride, dimenhydrinate, dimercaprol, dimethadione, dimethindene maleate, dimethisterone, dimethyl prostaglandin, dimethyl sulfoxide, dimethylhomospermine, dimiracetam, dimoxamine hydrochloride, dinoprost, dinoprostone, dioxadrol hydrochloride, dioxamycin, diphenhydramine citrate, diphenidol, diphenoxylate hydrochloride, diphenyl spiromustine, dipivefin hydrochloride, dipivefrin, dipliencyprone, diprafenone, dipropylnorspermine, dipyridamole, dipyrithione, dipyrone, dirithromycin, discodermolide, disobutamide, disofenin, disopyramide, disoxaril, disulfuram, ditekiren, divalproex sodium, dizocilpine maleate, dobutamine, docarpamine, docebenone, docetaxel, doconazole, docosanol, dofetilide, dolasetron, drotrecogin alfa, duloxetine hydrochloride, ebastine, ebiratide, ebrotidine, ebselen, ecabapide, ecabet, ecadotril, ecdisteron, echicetin, echistatin, echothiophate iodide, eclanamine maleate, eclazolast, ecomustine, econazole, ecteinascidin, edaravone, edatrexate, edelfosine, edifolone acetate, edobacomab, edoxudine, edrecolomab, edrophonium chloride, edroxyprogesteone acetate, efegatran, eflornithine, efonidipine, egualcen, elantrine, eleatonin, elemene, eletriptan, elgodipine, eliprodil, elsamitrucin, eltenae, elucaine, emalkalim, emedastine, emetine hydrochloride, emiglitate, emilium tosylate, emitefur, emoctakin, enadoline hydrochloride, enalapril, enalaprilat, enalkiren, enazadrem, encyprate, endralazine mesylate, endrysone, enflurane, englitazone, enilconazole, enisoprost, enlimomab, enloplatin, enofelast, enolicam sodium, enoxacin, enoxacin, enoxaparin sodium, enoxaparin sodium, enoximone, enpiroline phosphate, enprofylline, enpromate, entacapone, enterostatin, enviradene, enviroxime, ephedrine, epicillin, epimestrol, epinephrine, epinephryl borate, epipropidine, epirizole, epirubicin, epitetracycline hydrochloride, epithiazide, epoetin alfa, epoetin beta, epoprostenol, epoprostenol sodium, epoxymexrenone, epristeride, eprosartan, eptastigmine, equilenin, equilin, erbulozole, erdosteine, ergoloid mesylates, ergonovine maleate, ergotamine tartrate, ersentilide, ersofermin, erythritol, erythrityl tetranitrate, erythromycin, esmolol hydrochloride, esomeprazole, esorubicin hydrochloride, esproquin hydrochloride, estazolam, estradiol, estramustine, estramustine analogue, estrazinol hydrobromide, estriol, estrofurate, estrogen agonists, estrogen antagonists, estrogens, conjugated estrogens, esterified, estrone, estropipate, esuprone, etafedrine hydrochloride, etanidazole, etanterol, etarotene, etazolate hydrochloride, eterobarb, ethacizin, ethacrynate sodium, ethacrynic acid, ethambutol hydrochloride, ethamivan, ethanolamine oleate, ethehlorvynol, ether, ethinyl estradiol, ethiodized oil, ethionamide, ethonam nitrate, ethopropazine hydrochloride, ethosuximide, ethotoin, ethoxazene hydrochloride, ethybenztropine, ethyl chloride, ethyl dibunate, ethylestrenol, ethyndiol, ethynerone, ethynodiol diacetate, etibendazole, etidocaine, etidronate disodium, etidronic acid, etifenin, etintidine hydrochloride, etizolam, etodolac, etofenamate, etoformin hydrochloride, etomidate, etonogestrel, etoperidone hydrochloride, etoposide, etoprine, etoxadrol hydrochloride, etozolin, etrabamine, etretinate, etryptamine acetate, eucatropine hydrochloride, eugenol, euprocin hydrochloride, eveminomicin, exametazime, examorelin, exaprolol hydrochloride, exemestane, exetimibe, fadrozole, faeriefungin, famciclovir, famotidine, fampridine, fantof arone, fantridone hydrochloride, faropenem, fasidotril, fasudil, fazarabine, fedotozine, felbamate, felbinac, felodipine, felypressin, fenalamide, fenamole, fenbendazole, fenbufen, fencibutirol, fenclofenac, fenclonine, fenclorac, fendosal, fenestrel, fenethylline hydrochloride, fenfluramine hydrochloride, fengabine, fenimide, fenisorex, fenmetozole hydrochloride, fenmetramide, fenobam, fenoctimine sulfate, fenofibrate, fenoldopam, fenoprofen, fenoterol, fenpipalone, fenprinast hydrochloride, fenprostalene, fenquizone, fenretinide, fenspiride, fentanyl citrate, fentiazac, fenticlor, fenticonazole, fenyripol hydrochloride, fepradinol, ferpifosate sodium, ferristene, ferrixan, ferrous sulfate, ferumoxides, ferumoxsil, fetoxylate hydrochloride, fexofenadine, fezolamine fumarate, fiacitabine, fialuridine, fibrinogen, filgrastim, filipin, finasteride, flavodilol maleate, flavopiridol, flavoxate hydrochloride, flazalone, flecamide, flerobuterol, fleroxacin, flesinoxan, flestolol sulfate, fletazepam, flezelastine, flobufen, floctafenine, flomoxef, flordipine, florfenicol, florifenine, flosatidil, flosequinan, floxacillin, floxuridine, fluasterone, fluazacort, flubanilate hydrochloride, flubendazole, flucindole, flucloronide, fluconazole, flucytosine, fludalanine, fludarabine phosphate, fludazonium chloride, fludeoxyglucose, fludorex, fludrocortisone acetate, flufenamic acid, flufenisal, flumazenil, flumecinol, flumequine, flumeridone, flumethasone, flumetramide, flumezapine, fluminorex, flumizole, flumoxonide, flunarizine, flunidazole, flunisolide, flunitrazepam, flunixin, fluocalcitriol, fluocinolone acetonide, fluocinonide, fluocortin butyl, fluocortolone, fluorescein, fluorodaunorunicin hydrochloride, fluorodopa, fluoroformylone, fluoroquinolones, fluorometholone, fluorouracil, fluotracen hydrochloride, fluoxetine, fluoxymesterone, fluparoxan, fluperamide, fluperolone acetate, fluphenazine decanoate, flupirtine, fluprednisolone, fluproquazone, fluprostenol sodium, fluquazone, fluradoline hydrochloride, flurandrenolide, flurazepam hydrochloride, flurbiprofen, fluretofen, flurithromycin, fluorocitabine, fluorof amide, fluorogestone acetate, flurothyl, fluoroxene, fluspiperone, fluspirilene, fluticasone propionate, fluticasone furoate, flutrimazole, flutroline, fluvastatin, fluvastatin sodium, fluvoxamine, fluzinamide, folic acid, follicle regulatory protein, folliculostatin, fomepizole, fonazine mesylate, forasartan, forfenimex, forfenirmex, formestane, formocortal, formoterol, fosarilate, fosazepam, foscarnet sodium, fosfomycin, fosfonet sodium, fosinopril, fosinoprilat, fosphenyloin, fosquidone, fostedil, fostriecin, fotemustine, fuchsin, basic, fumoxicillin, fungimycin, furaprofen, furazolidone, furazolium chloride, furegrelate sodium, furobufen, furodazole, furosemide, fusidate sodium, fusidic acid, gabapentin, gadobenate dimeglumine, gadobenic acid, gadobutrol, gadodiamide, gadolinium texaphyrin, gadopentetate dimegiumine, gadoteric acid, gadoteridol, gadoversetamide, galantamine, galdansetron, galdansetron hydrochloride, gallamine triethiodide, gallium nitrate, gallopamil, galocitabine, gamfexine, gamolenic acid, ganciclovir, ganirelix, ganirelix acetate, gelatinase inhibitors, gemcadiol, gemcitabine, gemeprost, gemfibrozil, gentamicin sulfate, gentian violet, gepirone, gestaclone, gestodene, gestonorone caproate, gestrinone, gevotroline hydrochloride, girisopam, glaspimod, glaucocalyxin A, glemanserin, gliamilide, glibornuride, glicetanile sodium, gliflumide, glimepiride, glipizide, gloximonam, glucagon, glutapyrone, glutathione inhibitors, glutethimide, glyburide, glycopine, glycopril, glycopyrrolate, glyhexamide, glymidine sodium, glyoctamide, glyparamide, colloidal gold Au 198, gonadoctrinins, gonadorelin, gonadotropins, goserelin, gramicidin, granisetron, grepafloxacin, griseofulvin, guaiapate, guaithylline, guanabenz, guanabenz acetate, guanadrel sulfate, guancydine, guanethidine monosulfate, guanfacine hydrochloride, guanisoquin sulfate, guanoclor sulfate, guanoctine hydrochloride, guanoxabenz, guanoxan sulfate, guanoxyfen sulfate, gusperimus trihydrochloride, halazepam, halcinonide, halichondrin B, halobetasol propionate, halof antrine, halof antrine hydrochloride, halofenate, halofuginone hydrobromide, halomon, galopemide, galoperidol, halopredone, haloprogesterone, haloprogin, halothane, halquinols, hamycin, han menopausal gonadotropins, hatomamicin, hatomarubigin A, hatomarubigin B, hatomarubigin C, hatomarubigin D, heparin sodium, hepsulfam, heregulin, hetacillin, heteronium bromide, hexachlorophene: hydrogen peroxide, hexafluorenium bromide, hexamethylene bisacetamide, hexedine, hexobendine, hexoprenaline sulfate, hexylresorcinol, histamine phosphate, histidine, histoplasmin, histrelin, homatropine hydrobromide, hoquizil hydrochloride, human chorionic gonadotropin, hycanthone, hydralazine hydrochloride, hydralazine polistirex, hydrochlorothiazide, hydrocodone bitartrate, hydrocortisone, hydroflumethiazide, hydromorphone hydrochloride, hydroxyamphetamine hydrobromide, hydroxychloroquine sulfate, hydroxyphenamate, hydroxyprogesterone caproate, hydroxyurea, hydroxyzine hydrochloride, hymecromone, hyoscyamine, hypericin, ibafloxacin, ibandronic acid, ibogaine, ibopamine, ibudilast, ibufenac, ibuprofen, ibutilide fumarate, icatibant acetate, ichthammol, icotidine, idarubicin, idoxifene, idoxuridine, idramantone, iemefloxacin, iesopitron, ifetroban, ifosfamide, ilepeimide, illimaquinone, ilmofosine, ilomastat, ilonidap, iloperidone, iloprost, imafen hydrochloride, imazodan hydrochloride, imidapril, imidazenil, imidazoacridones, imidecyl iodine, imidocarb hydrochloride, imidoline hydrochloride, imidurea, imiloxan hydrochloride, imipenem, imipramine hydrochloride, imiquimod, immunostimulant peptides, impromidine hydrochloride, indacrinone, indapamide, indecamide hydrochloride, indeloxazine hydrochloride, indigotindisulfonate sodium, indinavir, indocyanine green, indolapril hydrochloride, indolidan, indometacin, indomethacin sodium, indoprofen, indoramin, indorenate hydrochloride, indoxole, indriline hydrochloride, infliximab, inocoterone, inogatran, inolimomab, inositol niacinate, insulin, insulin glargine, interferons, interferon beta-1a, interleukins, intrazole, intriptyline hydrochloride, iobenguane, iobenzamic acid, iobitridol, iocarmate meglumine, iocarmic acid, iocetamic acid, iodamide, iodine, iodipamide meglumine, iodixanol, iodoamiloride, iodoantipyrine, iodocholesterol, iododoxorubicin, iodohippurate sodium, iodopyracet, iodoquinol, iodoxamate meglumine, iodoxamie acid, ioglicic acid, iofetamine hydrochloride, iofratol, ioglucol, ioglucomide, ioglycamic acid, iogulamide, iohexyl, iomeprol, iomethin, iopamidol, iopanoic acid, iopentol, iophendylate, ioprocemic acid, iopromide, iopronic acid, iopydol, iopydone, iopyrol, iosefamic acid, ioseric acid, iosulamide meglumine, iosumetic acid, iotasul, iotetric acid, iothalamate sodium, iothalamic acid, iotriside, iotrolan, iotroxic acid, iotyrosine, ioversol, ioxagiate sodium, ioxaglate meglumine, ioxaglic acid, ioxilan, ioxotrizoic acid, ipazilide, ipenoxazone, ipidacrine, ipodate calcium, ipomeanol, 4-, ipratropium bromide, ipriflavone, iprindole, iprofenin, ipronidazole, iproplatin, iproxamine hydrochloride, ipsapirone, irbesartan, irinotecan, irloxacin, iroplact, irsogladine, irtemazole, isalsteine, isamoxole, isbogrel, isepamicin, isobengazole, isobutamben, isocarboxazid, isoconazole, isoetharine, isofloxythepin, isoflupredone acetate, isoflurane, isofluorophate, isohomohalicondrin B, isoleucine, isomazole hydrochloride, isomylamine hydrochloride, isoniazid, isopropamide iodide, isopropyl alcohol, isopropyl unoprostone, isoproterenol hydrochloride, isosorbide, isosorbide mononitrate, isotiquimide, isotretinoin, isoxepac, isoxicam, isoxsuprine hydrochloride, isradipine, itameline, itasetron, itazigrel, itopride, itraconazole, ivermectin, jasplakinolide, josamycin, kahalalide F, kalafungin, kanamycin sulfate, ketamine hydrochloride, ketanserin, ketazocine, ketazolam, kethoxal, ketipramine fumarate, ketoconazole, ketoprofen, ketorfanol, ketorolac, ketotifen fumarate, kitasamycin, labetalol hydrochloride, lacidipine, lacidipine, lactitol, lactivicin, laennec, lafutidine, lamellarin-n triacetate, lamifiban, lamivudine, lamotrigine, lanoconazole, lanperisone, lanreotide, lansoprazole, latanoprost, lateritin, laurocapram, lauryl isoquinolinium bromide, lavoltidine succinate, lazabemide, lecimibide, leinamycin, lemildipine, leminoprazole, lenercept, leniquinsin, lenograstim, lenperone, lentinan sulfate, leptin, leptolstatin, lercanidipine, lergotrile, lerisetron, letimide hydrochloride, letrazuril, letrozole, leucine, leucomyzin, leuprolide acetate, leuprolide, leuprorelin, levamfetamine succinate, levamisole, levdobutamine lactobionate, levcromakalim, levetiracetam, levobetaxolol, levobunolol, levobupivacaine, levocabastine, levocarnitine, levodopa, Ievodropropizine, levofloxacin, levofuraltadone, levoleucovorin calcium, levomethadyl acetate, levomethadyl acetate hydrochloride, levomoprolol, levonantradol hydrochloride, levonordefrin, levonorgestrel, levopropoxyphene napsylate, levopropylcillin potassium, levormeloxifene, levorphanol tartrate, levosimendan, levosulpiride, levothyroxine sodium, levoxadrol hydrochloride, lexipafant, lexithromycin, liarozole, libenzapril, lidamidine hydrochloride, lidocaine, lidofenin, lidoflazine, lifarizine, lifibrate, lifibrol, linarotene, lincomycin, linear polyamine analogue, linogliride, linopirdine, linotroban, linsidomine, lintitript, lintopride, liothyronine, liothyronine sodium, liotrix, lirexapride, lisinopril, lissoclinamide, lixazinone sulfate, lobaplatin, lobenzarit sodium, lobucavir, lodelaben, lodoxamide, lofemizole hydrochloride, lofentanil oxalate, lofepramine hydrochloride, lofexidine hydrochloride, lombricine, lomefloxacin, lomerizine, lometraline hydrochloride, lometrexol, lomitapide, lomofungin, lomoxicam, lomustine, lonapalene, lonazolac, lonidamine, loperamide hydrochloride, loracarbef, lorajmine hydrochloride, loratadine, lorazepam, lorbamate, lorcamide hydrochloride, loreclezole, lorglumide, lormetazepam, lomoxicam, lomoxicam, lortalamine, lorzafone, losartan, losigamone, losoxantrone, losulazine hydrochloride, loteprednol, lovastatin, loviride, loxapine, loxoribine, lubeluzole, lucanthone hydrochloride, lufironil, lurosetron mesylate, lurtotecan, luteinizing hormone, lutetium, lutrelin acetate, luzindole, lyapolate sodium, lycetamine, lydicamycin, lydimycin, lynestrenol, lypressin, lysine, lysofylline, lysostaphin, lytic peptides, maduramicin, mafenide, magainin 2 amide, magnesium salicylate, magnesium sulfate, magnolol, maitansine, malethamer, mallotochromene, mallotojaponin, malotilate, mangafodipir, manidipine, maniwamycin A, mannitol, mannostatin A, manumycin E, manumycin F, MAPK/ERK kinase (MEK) inhibitors, mapinastine, maprotiline, marimastat, masoprocol, maspin, massetolide, matrilysin inhibitors, maytansine, mazapertine succiniate, mazindol, mebendazole, mebeverine hydrochloride, mebrofenin, mebutamate, mecamylamine hydrochloride, mechlorethamine hydrochloride, meclocycline, meclofenamate sodium, mecloqualone, meclorisone dibutyrate, medazepam hydrochloride, medorinone, medrogestone, medroxalol, medroxyprogesterone, medrysone, meelizine hydrochloride, mefenamic acid, mefenidil, mefenorex hydrochloride, mefexamide, mefloquine hydrochloride, mefruside, megalomicin potassium phosphate, megestrol acetate, meglumine, meglutol, melengestrol acetate, melitracen hydrochloride, melphalan, memotine hydrochloride, menabitan hydrochloride, menoctone, menogaril, menotropins, meobentine sulfate, mepartricin, mepenzolate bromide, meperidine hydrochloride, mephentermine sulfate, mephenyloin, mephobarbital, mepivacaine hydrochloride, meprobamate, meptazinol hydrochloride, mequidox, meralein sodium, merbarone, mercaptopurine, mercufenol chloride, mercury, meropenem, mesalamine, meseclazone, mesoridazine, mesterolone, mestranol, mesuprine hydrochloride, metalol hydrochloride, metaproterenol polistirex, metaraminol bitartrate, metaxalone, meteneprost, meterelin, metformin, methacholine chloride, methacycline, methadone hydrochloride, methadyl acetate, methalthiazide, methamphetamine hydrochloride, methaqualone, methazolamide, methdilazine, methenamine, methenolone acetate, methetoin, methicillin sodium, methimazole, methioninase, methionine, methisazone, methixene hydrochloride, methocarbamol, methohexital sodium, methopholine, methotrexate, methotrimeprazine, methoxatone, methoxyflurane, methsuximide, methyclothiazide, methyl 10 palmoxirate, methylatropine nitrate, methylbenzethonium chloride, methyldopa, methyldopate hydrochloride, methylene blue, methylergonovine maleate, methylhistamine, R-alpha, methylinosine monophosphate, methylphenidate hydrochloride, methylprednisolone, methyltestosterone, methynodiol diacelate, methysergide, methysergide maleate, metiamide, metiapine, metioprim, metipamide, metipranolol, metizoline hydrochloride, metkephamid acetate, metoclopramide, metocurine iodide, metogest, metolazone, metopimazine, metoprine, metoprolol, metoquizine, metrifonate, metrizamide, metrizoate sodium, metronidazole, meturedepa, metyrapone, metyrosine, mexiletine hydrochloride, mexrenoate potassium, mezlocillin, mfonelic acid, mianserin hydrochloride, mibefradil, mibefradil dihydrochloride, mibolerone, michellamine B, miconazole, microcolin A, midaflur, midazolam hydrochloride, midodrine, mifepristone, mifobate, miglitol, milacemide, milameline, mildronate, milenperone, milipertine, milnacipran, milrinone, miltefosine, mimbane hydrochloride, minaprine, minaxolone, minocromil, minocycline, minoxidil, mioflazine hydrochloride, miokamycin, mipragoside, mirfentanil, mirimostim, mirincamycin hydrochloride, mirisetron maleate, mirtazapine, mismatched double stranded RNA, misonidazole, misoprostol, mitindomide, mitocarcin, mitocromin, mitogillin, mitoguazone, mitolactol, mitomalcin, mitomycin, mitonafide, mitosper, mitotane, mitoxantrone, mivacurium chloride, mivazerol, mixanpril, mixidine, mizolastine, mizoribine, moclobemide, modafinil, modaline sulfate, modecamide, moexipril, mof arotene, mofegiline hydrochloride, mofezolac, molgramostim, molinazone, molindone hydrochloride, molsidomine, mometasone, monatepil maleate, monensin, monoctanoin, montelukast sodium, montirelin, mopidamol, moracizine, morantel tartrate, moricizine, morniflumate, morphine, morphine sulfate, morrhuate sodium, mosapramine, mosapride, motilide, motretinide, moxalactam disodium, moxazocine, moxiraprine, moxnidazole, moxonidine, mumps skin test antigen, mustard anticancer agent, muzolimine, mycaperoxide B, mycophenolic acid, myriaporone, nabazenil, nabilone, nabitan hydrochloride, naboctate hydrochloride, nabumetone, n-acetyldinaline, nadide, nadifloxacin, nadolol, nadroparin calcium, nafadotride, nafamostat, nafarelin, nafcillin sodium, nafenopin, nafimidone hydrochloride, naflocort, nafomine malate, nafoxidine hydrochloride, nafronyl oxalate, naftifine hydrochloride, naftopidil, naglivan, nagrestip, nalbuphine hydrochloride, nalidixate sodium, nalidixic acid, nalmefene, nalmexone hydrochloride, naloxone/pentazocine, naltrexone, namoxyrate, nandrolone phenpropionate, nantradol hydrochloride, napactadine hydrochloride, napadisilate, napamezole hydyochloride, napaviin, naphazoline hydrochloride, naphterpin, naproxen, naproxol, napsagatran, naranol hydrochloride, narasin, naratriptan, nartograstim, nasaruplase, natamycin, nateplase, naxagolide hydrochloride, nebivolol, nebramycin, nedaplatin, nedocromil, nefazodone hydrochloride, neflumozide hydrochloride, nefopam hydrochloride, nelezaprine maleate, nemazoline hydrochloride, nemorubicin, neomycin palmitate, neostigmine bromide, neridronic acid, netilmicin sulfate, neutral endopeptidase, neutramycin, nevirapine, nexeridine hydrochloride, niacin, nibroxane, nicardipine hydrochloride, nicergoline, niclosamide, nicorandil, nicotinyl alcohol, nicotine, nifedipine, nifirmerone, nifluridide, nifuradene, nifuraldezone, nifuratel, nifuratrone, nifurdazil, nifurimide, nifurpirinol, nifurquinazol, nifurthiazole, nilutamide, nilvadipine, nimazone, nimodipine, niperotidine, niravoline, niridazole, nisamycin, nisbuterol mesylate, nisin, nisobamate, nisoldipine, nisoxetine, nisterime acetate, nitarsone, nitazoxamide, nitecapone, nitrafudam hydrochloride, nitralamine hydrochloride, nitramisole hydrochloride, nitrazepam, nitrendipine, nitrocycline, nitrodan, nitrofurantoin, nitrofurazone, nitroglycerin, nitromersol, nitromide, nitromifene citrate, nitrous oxide, nitroxide antioxidant, nitrullyn, nivazol, nivimedone sodium, nizatidine, noberastine, nocodazole, nogalamycin, nolinium bromide, nomifensine maleate, noracymethadol hydrochloride, norbolethone, norepinephrine bitartrate, norethindrone, norethynodrel, norfloxacin, norflurane, norgestimate, norgestomet, norgestrel, nortriptyline hydrochloride, noscapine, novobiocin sodium, N-substituted benzaimides, nufenoxole, nylestriol, nystatin, 06-benzylguanine, obidoxime chloride, ocaperidone, ocfentanil hydrochloride, ocinaplon, octanoic acid, octazamide, octenidine hydrochloride, octodrine, octreotide, octriptyline phosphate, ofloxacin, oformine, okicenone, olanzapine, oligonucleotides, olopatadine, olprinone, olsalazine, olsalazine sodium, olvanil, omeprazole, onapristone, ondansetron, ontazolast, oocyte maturation inhibitor, opipramol hydrochloride, oracin, orconazole nitrate, orgotein, orlislat, ormaplatin, ormetoprim, ornidazole, orpanoxin, orphenadrine citrate, osaterone, otenzepad, oxacillin sodium, oxagrelate, oxaliplatin, oxamarin hydrochloride, oxamisole, oxamniquine, oxandrolone, oxantel pamoate, oxaprotiline hydrochloride, oxaprozin, oxarbazole, oxatomide, oxaunomycin, oxazepam, oxcarbazepine, oxendolone, oxethazaine, oxetorone fumarate, oxfendazole, oxfenicine, oxibendazole, oxiconazole, oxidopamine, oxidronic acid, oxifungin hydrochloride, oxilorphan, oximonam, oximonam sodium, oxiperomide, oxiracetam, oxiramide, oxisuran, oxmetidine hydrochloride, oxodipine, oxogestone phenpropionate, oxolinic acid, oxprenolol hydrochloride, oxtriphylline, oxybutynin chloride, oxychlorosene, oxycodone, oxymetazoline hydrochloride, oxymetholone, oxymorphone hydrochloride, oxypertine, oxyphenbutazone, oxypurinol, oxytetracycline, oxytocin, ozagrel, ozolinone, paclitaxel, palauamine, paldimycin, palinavir, paliperidone, paliperidone palmitate, palmitoylrhizoxin, palmoxirate sodium, pamaqueside, pamatolol sulfate, pamicogrel, pamidronate disodium, pamidronic acid, panadiplon, panamesine, panaxytriol, pancopride, pancuronium bromide, panipenem, pannorin, panomifene, pantethine, pantoprazole, papaverine hydrochloride, parabactin, parachlorophenol, paraldehyde, paramethasone acetate, paranyline hydrochloride, parapenzolate bromide, pararosaniline pamoate, parbendazole, parconazole hydrochloride, paregoric, pareptide sulfate, pargyline hydrochloride, parnaparin sodium, paromomycin sulfate, paroxetine, parthenolide, partricin, paulomycin, pazelliptine, pazinaclone, pazoxide, pazufloxacin, pefloxacin, pegaspargase, pegorgotein, pelanserin hydrochloride, peldesine, peliomycin, pelretin, pelrinone hydrochloride, pemedolac, pemerid nitrate, pemetrexed, pemirolast, pemoline, penamecillin, penbutolol sulfate, penciclovir, penfluridol, penicillin G benzathine, penicillin G potassium, penicillin G procaine, penicillin G Sodium, penicillin V, penicillin V benzathine, penicillin V hydrabamine, penicillin V potassium, pentabamate, pentaerythritol tetranitrate, pentafuside, pentamidine, pentamorphone, bentamustine, pentapiperium methylsulfate, pentazocine, pentetic acid, pentiapine maleate, pentigetide, pentisomicin, pentizidone sodium, pentobarbital, pentomone, pentopril, pentosan, pentostatin, pentoxifylline, pentrinitrol, pentrozole, peplomycin sulfate, pepstatin, perflubron, perfof amide, perfosfamide, pergolide, perhexyline maleate, perillyl alcohol, perindopril, perindoprilat, perlapine, permethrin, perospirone, perphenazine, phenacemide, phenaridine, phenazinomycin, phenazopyridine hydrochloride, phenbutazone sodium glycerate, phencarbamide, phencyclidine hydrochloride, phendimetrazine tartrate, phenelzine sulfate, phenmetrazine hydrochloride, phenobarbital, phenoxybenzamine hydrochloride, phenprocoumon, phenserine, phensuccinal, phensuximide, phentermine, phentermine hydrochloride, phentolamine mesilate, phentoxifylline, phenyl aminosalicylate, phenylacetate, phenylalanine, phenylalanyl ketoconazole, phenylbutazone, phenylephrine hydrochloride, phenylpropanolamine hydrochloride, phenylpropanolamine polistirex, phenyramidol hydrochloride, phenyloin, phosphatase inhibitors, physostigmine, picenadol, picibanil, picotrin diolamine, picroliv, picumeterol, pidotimod, pifamine, pilocarpine, pilsicamide, pimagedine, pimetine hydrochloride, pimilprost, pimobendan, pimozide, pinacidil, pinadoline, pindolol, pinnenol, pinocebrin, pinoxepin hydrochloride, pioglitazone, pipamperone, pipazethate, pipecuronium bromide, piperacetazine, piperacillin sodium, piperamide maleate, piperazine, pipobroman, piposulfan, pipotiazine palmitate, pipoxolan hydrochloride, piprozolin, piquindone hydrochloride, piquizil hydrochloride, piracetam, pirandamine hydrochloride, pirarubicin, pirazmonam sodium, pirazolac, pirbenicillin sodium, pirbuterol acetate, pirenperone, pirenzepine hydrochloride, piretanide, pirfenidone, piridicillin sodium, piridronate sodium, piriprost, piritrexim, pirlimycin hydrochloride, pirlindole, pirmagrel, pirmenol hydrochloride, pirnabine, piroctone, pirodavir, pirodomast, pirogliride tartrate, pirolate, pirolazamide, piroxantrone hydrochloride, piroxicam, piroximone, pirprofen, pirquinozol, pirsidomine, prenylamine, pitavastatin, pituitary, posterior, pivampicillin hydrochloride, pivopril, pizotyline, placetin A, platinum compounds, platinum-triamine complex, plicamycin, plomestane, pobilukast edamine, podofilox, poisonoak extract, poldine methylsulfate, poliglusam, polignate sodium, polymyxin B sulfate, polythiazide, ponalrestat, porfimer sodium, porfiromycin, potassium chloride, potassium iodide, potassium permanganate, povidone-iodine, practolol, pralidoxime chloride, pramiracetam hydrochloride, pramoxine hydrochloride, pranlukast, pranolium chloride, prasugrel, pravadoline maleate, pravastatin, prazepam, praziquantel, prazosin, prazosin hydrochloride, prednazate, prednicarbate, prednimustine, prednisolone, prednisone, prednival, pregabalin, pregnenolone succiniate, prenalterol hydrochloride, pridefine hydrochloride, prifelone, prilocalne hydrochloride, prilosec, primaquine phosphate, primidolol, primidone, prinivil, prinomide tromethamine, prinoxodan, prizidilol hydrochloride, proadifen hydrochloride, probenecid, probicromil calcium, probucol, procainamide hydrochloride, procaine hydrochloride, procarbazine hydrochloride, procaterol hydrochloride, prochlorperazine, procinonide, proclonol, procyclidine hydrochloride, prodilidine hydrochloride, prodolic acid, prof adol hydrochloride, progabide, progesterone, proglumide, proinsulin human, proline, prolintane hydrochloride, promazine hydrochloride, promethazine hydrochloride, propafenone hydrochloride, propagermanium, propanidid, propantheline bromide, proparacaine hydrochloride, propatyl nitrate, propentofylline, propenzolate hydrochloride, propikacin, propiomazine, propionic acid, propionylcarnitine, propiram, propiram+paracetamol, propiverine, propofol, propoxycaine hydrochloride, propoxyphene hydrochloride, propranolol hydrochloride, propulsid, propyl bis-acridone, propylhexedrine, propyliodone, propylthiouracil, proquazone, prorenoate potassium, proroxan hydrochloride, proscillaridin, prostalene, prostratin, protamine sulfate, protegrin, protirelin, protosufloxacin, protriptyline hydrochloride, proxazole, proxazole citrate, proxicromil, proxorphan tartrate, prulifloxacin, pseudoephedrine hydrochloride, desloratadine/pseudoephedrine sulfate, puromycin, purpurins, pyrabrom, pyrantel, pamoate, pyrazinamide, pyrazofurin, pyrazoloacridine, pyridostigmine bromide, pyrilamine maleate, pyrimethamine, pyrinoline, pyrithione sodium, pyrithione zinc, pyrovalerone hydrochloride, pyroxamine maleate, pyrrocaine, pyrroliphene hydrochloride, pyrroinitrin, pyrvinium pamoate, quadazocine mesylate, quazepam, quazinone, quazodine, quazolast, quetiapine, quiflapon, quinagolide, quinaldine blue, quinapril, quinaprilat, quinazosin hydrochloride, quinbolone, quinctolate, quindecamine acetate, quindonium bromide, quinelorane hydrochloride, quinestrol, quinfamide, quingestanol acetate, quingestrone, quinidine gluconate, quinielorane hydrochloride, quinine sulfate, quinpirole hydrochloride, quinterenol sulfate, quinuclium bromide, quinupristin, quipazine maleate, rabeprazole sodium, racephenicol, racepinephrine, rafoxamide, ralitoline, raloxifene, raltitrexed, ramatroban, ramipril, ramoplanin, ramosetron, ranelic acid, ranimycin, ranitidine, ranolazine, rauwolfia serpentina, recainam, recainam hydrochloride, reclazepam, regavirumab, regramostim, relaxin, relomycin, remacemide hydrochloride, remifentanil hydrochloride, remiprostol, remoxipride, repirinast, repromicin, reproterol hydrochloride, reserpine, resinferatoxin, resorcinol, retelliptine demethylated, reticulon, reviparin sodium, revizinone, rhenium re 186 etidronate, rhizoxin, ribaminol, ribavirin, riboprine, ribozymes, ricasetron, ridogrel, rifabutin, rifametane, rifamexil, rifamide, rifampin, rifapentine, rifaximin, retinamide, rilopirox, riluzole, rimantadine, rimcazole hydrochloride, rimexolone, rimiterol hydrobromide, rimoprogin, riodipine, rioprostil, ripazepam, ripisartan, risedronate sodium, risedronic acid, risocaine, risotilide hydrochloride, rispenzepine, risperdal, risperidone, ritanserin, ritipenem, ritodrine, ritolukast, ritonavir, rizatriptan benzoate, rocastine hydrochloride, rocuronium bromide, rodocaine, roflurane, rogletimide, rohitukine, rokitamycin, roletamicide, rolgamidine, rolicyprine, rolipram, rolitetracycline, rolodine, romazarit, romurtide, ronidazole, ropinirole, ropitoin hydrochloride, ropivacaine, ropizine, roquinimex, rosaramicin, rosoxacin, rotoxamine, rosuvastatin, roxaitidine, roxarsone, roxindole, roxithromycin, rubiginone Bl, ruboxyl, rufloxacin, rupatidine, rutamycin, ruzadolane, sabeluzole, safingol, safironil, saintopin, salbutamol, salcolex, salethamide maleate, salicyl alcohol, salicylamide, salicylate meglumine, salicylic acid, salmeterol, salnacediin, salsalate, sameridine, sampatrilat, sancycline, sanfetrinem, sanguinarium chloride, saperconazole, saprisartan, sapropterin, saquinavir, sarafloxacin hydrochloride, saralasin acetate, SarCNU, sarcophytol A, sargramostim, sarmoxicillin, sarpicillin, sarpogrelate, saruplase, saterinone, satigrel, satumomab pendetide, Schick test control, scopafungin, scopolamine hydrobromide, scrazaipine hydrochloride, sdi 1 mimetics, secalciferol, secobarbital, seelzone, seglitide acetate, selegiline, selegiline hydrochloride, selenium sulfide, selenomethionine se 75, selfotel, sematilide, semduramicin, semotiadil, semustine, sense oligonucleotides, sepazonium chloride, seperidol hydrochloride, seprilose, seproxetine hydrochloride, seractide acetate, sergolexole maleate, serine, sermetacin, sermorelin acetate, sertaconazole, sertindole, sertraline, setiptiline, setoperone, sevirumab, sevoflurane, sezolamide, sibopirdine, sibutramine hydrochloride, signal transduction inhibitors, silandrone, sildenafil, silipide, silteplase, silver nitrate, simendan, simtrazene, simvastatin, sinefungin, sinitrodil, sinnabidol, sipatrigine, sirolimus, sisomicin, sitogluside, sizofuran, sobuzoxane, sodium amylosulfate, sodium iodide, sodium nitroprusside, sodium oxybate, sodium phenylacetate, sodium salicylate, solegabron, solverol, solypertine tartrate, somalapor, somantadine hydrochloride, somatomedin B, somatomedin C, somatrem, somatropin, somenopor, somidobove, sonermin, sorbinil, sorivudine, sotalol, soterenol hydrochloride, sparfloxacin, sparfosate sodium, sparfosic acid, sparsomycin, sparteine sulfate, spectinomycin hydrochloride, spicamycin D, spiperone, spiradoline mesylate, spiramycin, spirapril hydrochloride, spiraprilat, spirogermanium hydrochloride, spiromustine, spironolactone, spiroplatin, spiroxasone, splenopentin, spongistatin 1, sprodiamide, squalamine, stallimycin hydrochloride, stannous pyrophosphate, stannous sulfur colloid, stanozolol, statolon, staurosporine, stavudine, steffimycin, stenbolone acetate, stepronin, stilbazium iodide, stilonium iodide, stipiamide, stiripentol, stobadine, streptomycin sulfate, streptonicozid, streptonigrin, streptozocin, stromelysin inhibitors, strontium chloride Sr 89, succibun, succimer, succinylcholine chloride, sucralfate, sucrosofate potassium, sudoxicam, sufentanil, sufotidine, sulazepam, sulbactam pivoxil, sulconazole nitrate, sulfabenz, sulfabenzamide, sulfacetamide, sulfacytine, sulfadiazine, sulfadoxine, sulfalene, sulfamerazine, sulfameter, sulfamethazine, sulfamethizole, sulfamethoxazole, sulfamonomethoxine, sulfamoxole, sulfanilate zinc, sulfanitran, sulfasalazine, sulfasomizole, sulfazamet, sulfinalol hydrochloride, sulfinosine, sulfinpyrazone, sulfisoxazole, sulfomyxin, sulfonterol hydrochloride, sulfoxamine, sulinldac, sulmarin, sulnidazole, suloctidil, sulofenur, sulopenem, suloxifen oxalate, sulpiride, sulprostone, sultamicillin, sulthiame, sultopride, sulukast, sumarotene, sumatriptan, suncillin sodium, suproclone, suprofen, suradista, suramin, surfomer, suricamide maleate, suritozole, suronacrine maleate, suxemerid sulfate, swainsonine, symakalim, symclosene, symetine hydrochloride, synthetic glycosaminoglycans, tadalafil, taciamine hydrochloride, tacrine hydrochloride, tacrolimus, talampicillin hydrochloride, taleranol, talisomycin, tallimustine, talmetacin, talniflumate, talopram hydrochloride, talosalate, tametraline hydrochloride, tamoxifen, tampramine fumarate, tamsulosin hydrochloride, tandamine hydrochloride, tandospirone, tapgen, taprostene, tasosartan, tauromustine, taxane, taxoid, tazadolene succinate, tazanolast, tazarotene, tazifylline hydrochloride, tazobactam, tazofelone, tazolol hydrochloride, tebufelone, tebuquine, technetium Tc 99 m bicisate, teclozan, tecogalan sodium, teecleukin, teflurane, tegafur, tegretol, teicoplanin, telenzepine, tellurapyrylium, telmesteine, telmisartan, telomerase inhibitors, teloxantrone hydrochloride, teludipine hydrochloride, temafloxacin hydrochloride, tematropium methyl sulfate, temazepam, temelastine, temocapril, temocillin, temoporfin, temozolomide, tenofovir, tenidap, teniposide, tenosal, tenoxicam, tepirindole, tepoxalin, teprotide, terazosin, terbinafine, terbutaline sulfate, terconazole, terfenadine, terflavoxate, terguride, teriparatide acetate, terlakiren, terlipressin, terodiline, teroxalene hydrochloride, teroxirone, tertatolol, tesicam, tesimide, testolactone, testosterone, tetracaine, tetrachlorodecaoxide, tetracycline, tetrahydrozoline hydrochloride, tetramisole hydrochloride, tetrazolast meglumine, tetrazomine, tetrofosmin, tetroquinone, tetroxoprim, tetrydamine, thaliblastine, thalidomide, theofibrate, theophylline, thiabendazole, thiamiprine, thiamphenicol, thiamylal, thiazesim hydrochloride, thiazinamium chloride, thiazolidinedione, thiethylperazine, thimerfonate sodium, thimerosal, thiocoraline, thiofedrine, thioguanine, thiomarinol, thiopental sodium, thioperamide, thioridazine, thiotepa, thiothixene, thiphenamil hydrochloride, thiphencillin potassium, thiram, thozalinone, threonine, thrombin, thrombopoietin, thrombopoietin mimetic, thymalfasin, thymopoietin receptor agonist, thymotrinan, thyromedan hydrochloride, thyroxine, tiacrilast, tiacrilast sodium, tiagabine, tiamenidine, tianeptine, tiapafant, tiapamil hydrochloride, tiaramide hydrochloride, tiazofurin, tibenelast sodium, tibolone, tibric acid, ticabesone propionate, ticarbodine, ticarcillin cresyl sodium, ticlatone, ticlopidine, ticrynafen, tienoxolol, tifurac sodium, tigemonam dicholine, tigestol, tiletamine hydrochloride, tilidine hydrochloride, tilisolol, tilnoprofen arbamel, tilorone hydrochloride, tiludronate disodium, tiludronic acid, timefurone, timobesone acetate, timolol, tinethyl etiopurpurin, tinabinol, timidazole, tinzaparin sodium, tioconazole, tiodazosin, tiodonium chloride, tioperidone hydrochloride, tiopinac, tiospirone hydrochloride, tiotidine, tiotropium bromide, tioxidazole, tipentosin hydrochloride, tipredane, tiprenolol hydrochloride, tiprinast meglumine, tipropidil hydrochloride, tiqueside, tiquinamide hydrochloride, tirandalydigin, tirapazamine, tirilazad, tirofiban, tiropramide, titanocene dichloride, tixanox, tixocortol pivalate, tizanidine hydrochloride, tobramycin, tocamide, tocamphyl, tofenacin hydrochloride, tolamolol, tolazamide, tolazoline hydrochloride, tolbutamide, tolcapone, tolciclate, tolfamide, tolgabide, lamotrigine, tolimidone, tolindate, tolmetin, tolnaftate, tolpovidone, tolpyrramide, tolrestat, tomelukast, tomoxetine hydrochloride, tonazocine mesylate, topiramate, topotecan, topotecan hydrochloride, topsentin, topterone, toquizine, torasemide, toremifene, torsemide, tosifen, tosufloxacin, totipotent stem cell factor, tracazolate, trafermin, tralonide, tramadol hydrochloride, tramazoline hydrochloride, trandolapril, tranexamic acid, tranilast, transcamide, translation inhibitors, trastuzumab, traxanox, trazodone hydrochloride, trazodone-hcl, trebenzomine hydrochloride, trefentanil hydrochloride, treloxinate, trepipam maleate, trestolone acetate, tretinoin, triacetin, triacetyluridine, triafungin, triamcinolone, triampyzine sulfate, triamterene, triazolam, tribenoside, tricaprilin, tricetamide, trichlormethiazide, trichohyalin, triciribine, tricitrates, triclofenol piperazine, triclofos sodium, triclonide, trientine, trifenagrel, triflavin, triflocin, triflubazam, triflumidate, trifluoperazine hydrochloride, trifluperidol, triflupromazine, triflupromazine hydrochloride, trifluridine, trihexyphenidyl hydrochloride, trilostane, trimazosin hydrochloride, trimegestone, trimeprazine tartrate, trimethadione, trimethaphan camsylate, trimethobenzamide hydrochloride, trimethoprim, trimetozine, trimetrexate, trimipramine, trimoprostil, trimoxamine hydrochloride, triolein, trioxifene mesylate, tripamide, tripelennamine hydrochloride, triprolidine hydrochloride, triptorelin, trisulfapyrimidines, troclosene potassium, troglitazone, trolamine, troleandomycin, trombodipine, trometamol, tropanserin hydrochloride, tropicamide, tropine ester, tropisetron, trospectomycin, trovafloxacin, trovirdine, tryptophan, tuberculin, tubocurarine chloride, tubulozole hydrochloride, tucarcsol, tulobuterol, turosteride, tybamate, tylogenin, tyropanoate sodium, tyrosine, tyrothricin, tyrphostins, ubenimex, uldazepam, undecylenic acid, uracil mustard, urapidil, urea, uredepa, uridine triphosphate, urofollitropin, urokinase, ursodiol, valaciclovir, valine, valnoctamide, valproate sodium, valproic acid, valsartan, vamicamide, vanadeine, vancomycin, vaminolol, vapiprost hydrochloride, vapreotide, vardenafil, variolin B, vasopressin, vecuronium bromide, velaresol, velnacrine maleate, venlafaxine, veradoline hydrochloride, veramine, verapamil hydrochloride, verdins, verilopam hydrochloride, verlukast, verofylline, veroxan, verteporfin, vesnarinone, vexibinol, vidarabine, vigabatrin, viloxazine hydrochloride, vinblastine sulfate, vinburnine citrate, vincofos, vinconate, vincristine sulfate, vindesine, vindesine sulfate, vinepidine sulfate, vinglycinate sulfate, vinleurosine sulfate, vinorelbine, vinpocetine, vintoperol, vinxaltine, vinzolidine sulfate, viprostol, virginiamycin, viridofulvin, viroxime, vitaxin, volazocine, voriconazole, vorozole, voxergolide, warfarin sodium, xamoterol, xanomeline, xanoxate sodium, xanthinol niacinate, xemilofiban, xenalipin, xenbucin, xilobam, ximoprofen, xipamide, xorphanol mesylate, xylamidine tosylate, xylazine hydrochloride, xylometazoline hydrochloride, xylose, yangambin, zabicipril, zacopride, zafirlukast, zalcitabine, zaleplon, zalospirone, zaltidine hydrochloride, zaltoprofen, zanamivir, zankiren, zanoterone, zantac, zarirlukast, zatebradine, zatosetron, zatosetron maleate, zenarestat, zenazocine mesylate, zeniplatin, zeranol, zidometacin, zidovudine, zifrosilone, zilantel, zilascorb, zileuton, zimeldine hydrochloride, zinc undecylenate, zindotrine, zinoconazole hydrochloride, zinostatin, zinterol hydrochloride, zinviroxime, ziprasidone, zobolt, zofenopril calcium, zofenoprilat, zolamine hydrochloride, zolazepam hydrochloride, zoledronie acid, zolertine hydrochloride, zolmitriptan, Zolpidem, zomepirac sodium, zometapine, zoniclezole hydrochloride, zonisamide, zopiclone, zopolrestat, zorbamyciin, zorubicin hydrochloride, zotepine, zucapsaicin, reglitazar, netoglitazone, tesaglitazar, demethyl asteriquinone, pharmaceutically acceptable salts thereof e.g., Zn, Fe, Mg, K, Na, F, Cl, Br, I, acetate, diacetate, nitrate, nitrite, sulfate, sulfite, phosphate, and phosphite salts, pharmaceutically acceptable forms thereof with acid associates (e.g. HCl), or any combination thereof.

Suitable antibiotics for use in conjunction with the present disclosure may include, but are not limited to, to [beta]-lactam antibiotics (e.g., benzathine penicillin, benzylpenicillin (penicillin G), phenoxymethylpenicillin (penicillin V), procaine penicillin, methicillin, oxacillin, nafcillin, cloxacillin, dicloxacillin, flucloxacillin, temocillin, amoxicillin, ampicillin, co-amoxiclav (a moxicillin+clavulanic acid), azlocillin, carbenicillin, ticarcillin, mezlocillin, piperacillin, cephalosporin, cephalexin, cephalothin, cefazolin, cefaclor, cefuroxime, cefamandole, cefotetan, cefoxitin, ceftriaxone, cefotaxime, cefpodoxime, cefixime, ceftazidime, cefepime, cefpirome, carbapenem, imipenem (with cilastatin), meropenem, ertapenem, faropenem, doripenem, aztreonam, tigemonam, nocardicin A, tabtoxinine- -lactam, clavulanic acid, tazobactam, and sulbactam; aminoglycoside antibiotics (e.g., aminoglycoside, amikacin, apramycin, arbekacin, astromicin, bekanamycin, capreomycin, dibekacin, dihydrostreptomycin, elsamitrucin, G418, gentamicin, hygromycin B, isepamicin, kanamycin, kasugamycin, micronomicin, neomycin, netilmicin, paromomycin sulfate, ribostamycin, sisomicin, streptoduocin, streptomycin, tobramycin, verdamicin; sulfonamides such as sulfamethoxazole, sulfisomidine (also known as su lfaisodimidine), sulfacetamide, sulfadoxine, dichlorphenamide (DCP), and dorzolamide); quinolone antibiotics (e.g., cinobac, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, garenoxacin, and delafloxacin); oxazolidone antibiotics (e.g., linezolid, torezolid, eperezolid, posizolid, and radezolid), or any combination thereof.

Suitable antifungals for use in conjunction with the present disclosure may include, but are not limited to, polyene antifungals (e.g., natamycin, rimocidin, filipin, nystatin, amphotericin B, candicin, and hamycin; imidazole antifungals such as miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazole, terconazole, and albaconazole), thiazole antifungals (e.g., abafungin), allylamine antifungals (e.g., terbinafine, naftifine and butenafine), echinocandin antifungals (e.g., anidulafungin, caspofungin, and micafungin), polygodial, benzoic acid, ciclopirox, tolnaftate, undecylenic acid, flucytosine, 5-fluorocytosine, griseofulvin, haloprogin, and any combination thereof. Suitable active biologicals for use in conjunction with the present disclosure may include, but are not limited to, hormones (synthetic or natural and patient derived or otherwise), DNAs (synthetic or natural and patient derived or otherwise), RNAs (synthetic or natural and patient derived or otherwise), siRNAs (synthetic or natural and patient derived or otherwise), proteins and peptides (e.g., albumin, atrial natriuretic factor, renin, superoxide dismutase, bacitracin, bestatin, cydosporine, delta sleep-inducing peptide (DSIP), endorphins, glucagon, gramicidin, melanocyte inhibiting factors, neurotensin, oxytocin, somostatin, terprotide, serum thymide factor, thymosin, DDAVP, dermorphin, Met-enkephalin, peptidoglycan, satietin, thymopentin, fibrin degradation product, des-enkephalin-a-endorphin, gonadotropin releasing hormone, leuprolide, a-MSH, and metkephamid), enzymes, nucleotides, oligionucleotides, antibodies, monoclonal antibodies, growth factors (e.g., epidermal growth factor (EGF), fibroblast growth factors, basic fibroblast growth factor (bFGF), nerve growth factor (NGF), bone derived growth factor (BDGF), transforming growth factors, transforming growth factor-[beta] [iota] (TGF-[beta] [Iota]), and human growth gormone (hGH)), viral surface antigens (e.g., adenoviruses, epstein-barr virus, hepatitis A virus, hepatitis B virus, herpes viruses, HIV-1, HIV-2, HTLV-III, influenza viruses, Japanese encephalitis virus, measles virus, papilloma viruses, paramyxoviruses, polio virus, rabies virus, rubella virus, vaccinia (smallpox) viruses, and yellow fever virus), bacterial surface antigens (e.g., bordetella pertussis, helicobacter pylori, Clostridium tetani, corynebacterium diphtheria, escherichia coli, haemophilus influenza, klebsiella species, legionella pneumophila, mycobacterium bovis, mycobacterium leprae, mycrobacterium tuberculosis, neisseria gonorrhoeae, neisseria meningitidis, proteus species, pseudomonas aeruginosa, salmonella species, shigella species, staphylococcus aureus, streptococcus pyogenes, vibrio cholera, and yersinia pestis), parasite surface antigens (e.g., Plasmodium vivax—malaria, Plasmodium falciparum—malaria, Plasmodium ovale—malaria, Plasmodium malariae—malaria, leishmania tropica—leishmaniasis, leishmania donovani, leishmaniasis, leishmania branziliensis—leishmaniasis, trypanosoma rhodescense—sleeping sickness, trypanosoma gambiense—sleeping sickness, trypanosoma cruzi—Chagas' disease, schistosoma mansoni—schistosomiasis, schistosomoma haematobium—schistomiasis, schistosoma japonicum—shichtomiasis, trichinella spiralis—trichinosis, stronglyloides duodenale—hookworm, ancyclostoma duodenale—hookworm, necator americanus—hookworm, wucheria bancrofti—filariasis, brugia malaya—filariasis, loa loa—filariasis, dipetalonema perstaris—filariasis, dracuncula medinensis—filariasis, and onchocerca volvulus—filariasis), immunogobulins (e.g., IgG, IgA, IgM, antirabies immunoglobulin, and antivaccinia immunoglobulin), and any combination thereof.

Suitable antitoxins for use in conjunction with the present disclosure may include, but are not limited to, botulinum antitoxin, diphtheria antitoxin, gas gangrene antitoxin, tetanus antitoxin, or any combination thereof.

Suitable antigens for use in conjunction with the present disclosure may include, but are not limited to, foot and mouth disease, hormones and growth factors (e.g., follicle stimulating hormone, prolactin, angiogenin, epidermal growth factor, calcitonin, erythropoietin, thyrotropic releasing hormone, insulin, growth hormones, insulin-like growth factors 1 and 2, skeletal growth factor, human chorionic gonadotropin, luteinizing hormone, nerve growth factor, adrenocorticotropic hormone (ACTH), luteinizing hormone releasing hormone (LHRH), parathyroid hormone (PTH), thyrotropin releasing hormone (TRH), vasopressin, cholecystokinin, and corticotropin releasing hormone), cytokines (e.g., interferons, interleukins, colony stimulating factors, and tumor necrosis factors: fibrinolytic enzymes, such as urokinase, kidney plasminogen activator), clotting factors (e.g., Protein C, Factor VIII, Factor IX, Factor VII and Antithrombin III), or any combination thereof.

Suitable nutritional supplements for use in conjunction with the present disclosure may include, but are not limited to, vitamins, minerals, probiotics, herbs, botanicals, aminoacids, steroids, and the like.

Suitable imaging agents for use in conjunction with the present disclosure may include, but are not limited to, iron oxide, gadolinium ions, iodine, perfluorocarbons, radioisotopes, and the like.

In particularly preferred embodiments, the active agent may be an analgesic such as paracetamol; an antibiotic such as amoxicillin; a receptor agonist or antagonist such as alfuzosin HCl, atenolol or losartan; an anti-viral drug such as acyclovir; an anthelmintic such as praziquantel; or a beta blocker such as propranolol.

The active agent is incorporated in the gel composition according to the present disclosure. Preferably, the active agent (component (b) of the composition) may comprise up to about 0.25%, or at least about 0.25%, or at least about 0.5%, or at least about 1%, or at least about 2%, or at least about 5%, or at least about 10%, or at least about 12%, or at least about 15%, or at least about 20%, or at least about 25%, or up to about 25%, or up to about 30%, or at least about 30%, or at least about 35%, or up to about 35%, or up to about 40% or at least about 40%, or up to about 45% or at least about 45%, or up to about 50% by weight of said gel composition.

The active agent in the gel composition of the present disclosure may be conventionally formulated for immediate absorption of the active ingredient upon release from the gel composition. In particular, the active agent may be formulated as a powder, as pellets including coated pellets, as particles, nanoparticles or granules, or as a liquid or gel. Alternatively, the active agent may be formulated so as to enable controlled, sustained or modified release of the active ingredient over a period of time. For example, the active agent may be provided in a prepackaged form for controlled, sustained or modified release of the active ingredient, such as capsules, pellets, non pareils, microballoons, microspheres or beads. Release of the active ingredient from these prepackaged forms may be controlled by a diffusion polymer layer. The diffusion polymer layer may comprise polymers with low water solubility or polymers which are insoluble in water. Optionally, the polymers may have some solubility in a hydrophobic environment. To prevent dissolution rate changes or active degradation over time, the diffusion polymer layer may comprise an extra layer of a hydrophilic polymer such as HPMC acting as an insulating layer between the hydrophobic polymeric film and the lipidic environment.

The gel composition of the present disclosure may comprise more than one active agent. In this case, the active agents may be separately prepackaged or preformulated for independent release and delivery, or may be prepackaged or preformulated together.

The active agent may be dispersed, solubilised or suspended in the gel composition. Where the active agent is formulated as particles or microparticles or nanoparticles, the active agent may be dispersed with an even or uneven particle size distribution.

In some especially preferred embodiments, component (a) of the gel composition of the present disclosure may comprise ethylcellulose 50cPs gelled with Labrafac™, Peceol™ or Maisine™ in a w/w ratio of about 4:96 (4% w/w organogelator), and the hard wax or wax-like additive of component (b) may comprise Geleol™, Gelucire®, Suppocire® AM and/or Suppocire® AML. The active agent of component (c) may comprise paracetamol, praziquantel, amoxicillin or alfuzosin, and may be provided in powder form or as pellets.

In particular, the hydrophobic or amphiphilic liquid in the gel composition of the present disclosure may advantageously comprise Maisine™ and the hard wax or wax-like additive may advantageously comprise Suppocire®.

The gel composition of the present disclosure may optionally comprise:
15-90% w/w, or preferably 30-80% w/w, hydrophobic or amphiphilic liquid;
0.2-15% w/w, or preferably 1-4% w/w, organogelator;
0.1-50% w/w, or preferably 10-50% w/w, active agent; and
2-70% w/w, preferably 10-60% w/w, hard wax or wax-like additive;
wherein the sum of these percentages is 100%.

The gel composition of the present disclosure may comprise additional ingredients and additives. In particular, the composition may include a surfactant, such as lecithin, for example in a quantity up to about 5% by weight of the composition. Lecithin may help to improve the dispersion of a non-soluble drug and/or may help to keep the drug in suspension in order to improve control of the drug release rate. The composition may comprise preservatives and/or antioxidative agents such as BHA (butyl hydroxyl anisole) or BHT (butyl hydroxyl toluene), and/or may comprise flavouring and/or colouring and/or taste masking agents, such as orange, grapefruit and/or mint flavours or sweeteners such as sucralose.

The gel composition of the present disclosure may accordingly optionally comprise:
15-90% w/w, or preferably 30-80% w/w, hydrophobic or amphiphilic liquid;
0.2-15% w/w, or preferably 1-4% w/w, organogelator;
0.1-50% w/w, or preferably 10-50% w/w, active agent;
2-70% w/w, preferably 10-60% w/w, hard wax or wax-like additive; and
up to 10% w/w, or preferably up to 5% w/w, of additional ingredients and additives, optionally selected from surfactants, preservatives, anti-oxidants, flavorants, colorants, taste masking agents and sweeteners;
   wherein the sum of these percentages is 100%.

Suitably, the composition of the present disclosure may be formulated to enhance its buoyancy in gastric fluid and to improve control of the release of the active agent from the composition. Factors which may affect the buoyancy of the composition and the active agent release characteristics include the type of hydrophobic or amphiphilic liquid; the type and grade of polymeric organogelator; the relative quantities of organogelator and hydrophobic or amphiphilic liquid; the type and quantity of active agent; the formulation of the active agent; the inclusion of additional ingredients; and so on.

Desirably, these factors may be selected such that the composition is buoyant in gastric fluid, and that the active agent is fully released from the composition within 24 hours, preferably within 20 hours, or within 18 hours following in vivo administration to a patient. Suitably, these factors may be selected such that the active agent is 80% released from the composition within 2-20, or 4-20, or 6-20 hours following in vivo administration, and/or is fully released from the composition within 5-20 or 7-20 or 10-20 hours following in vivo administration.

Advantageously, the composition of the present disclosure may be suitable for administration by way of a sachet, such as a squeezable sachet or monodose sachet; or a bottle; or a tube; or a dosing syringe; or a bottle with a dosing pump. Thus, a further aspect of the present disclosure comprehends a dosage form which is a sachet, bottle, tube, multidose dosing syringe, or a bottle with a dosing pump comprising the composition of the present disclosure. Suitably, the dosage form may be adapted to enable convenient delivery and administration of a predetermined quantity of the composition, representing a dose of said active agent. Thus, the dosage form may be a single dose sachet containing a predetermined quantity of the composition; or a bottle, tube or syringe that is configured to dispense a predetermined quantity of the composition representing a dose or multiple doses of the active agent. The dosage form may preferably be made from aluminium, plastic, polymeric material, glass, or any other suitable inert material, or a combination of any of these.

In a further aspect, the present disclosure provides a method for administering an active agent to or through the upper gastrointestinal tract, in particular the stomach, of a human or animal patient, comprising orally administering to the patient a gel composition according to the present disclosure which comprises said active agent. Preferably, the patient is in a non-fasting state, which means that the stomach of the patient is not empty. Suitably, the patient may have eaten or may be instructed to eat food no more than 2 hours, preferably no more than 1 hour, before oral administration of the composition. The patient may eat or be instructed to eat food just before or in conjunction with oral administration of the composition.

In a further aspect, the present disclosure provides a gel composition comprising an active agent, in accordance with the present disclosure, for use in gastric delivery of the active agent to a human or animal patient. The present disclosure further provides a gel composition comprising an active agent, in accordance with the present disclosure, for use in administering said active agent to or through the upper gastrointestinal tract, in particular the stomach, of a human or animal patient. The present disclosure further provides a gel composition in accordance with the present disclosure for use in oral administration to a human or animal patient. Preferably, the patient is in a non-fasting state, which means that the stomach of the patient is not empty. Suitably, the patient may have eaten or may be instructed to eat food no more than 2 hours, preferably no more than 1 hour, before oral administration of the composition. The patient may eat or be instructed to eat food just before or in conjunction with oral administration of the composition.

In yet a further aspect, the present disclosure provides the use of a composition in accordance with the present disclosure in the manufacture of a medicament for gastric delivery of said active agent. The medicament may be a medicament for treating a disease or condition listed above.

A range of different oleogels, active agents and hard wax ingredients may be used in compositions according to the present disclosure. The density of the composition obtained is significant, as the composition must be capable of floating on the contents of the stomach in order to enable gastric retention. For illustrative purposes, Table 1 below shows the density of a number of oily bases useful in the present disclosure, of oleogels formed from a selection of these oily bases; and of a number of these oleogels including an active agent.

TABLE 1

| | | | Density |
|---|---|---|---|
| Oil base | Polymer | Active agent | (g/mL) |
| Capryol 90 | | | 0.939 |
| Labrafac PG | | | 0.906 |
| Labrafil 1944 CS | | | 0.933 |
| Lauroglycol | | | 0.909 |
| Maisine 35-1 | | | 0.934 |
| Peceol | | | 0.935 |
| Plurol oleique | | | 0.969 |
| Capryol 90 | 4% PEO WSRN-80 | | 0.946 |
| Lauroglycol 90 | 4% PEO WSRN-80 | | 0.941 |
| Maisine 35-1 | 4% PEO WSRN-80 | | 0.967 |
| Peceol | 4% PEO WSRN-80 | | 0.929 |
| Plurol oleique | 4% Lutrol F127 | | 0.997 |
| Capryol | 4% Etc 50 cP | Acyclovir 100 mg | 1.039 |
| Lauroglycol | 4% Etc-50 cP | Acyclovir 100 mg | 1.042 |

Again for illustrative purposes, Table 2 lists the composition of various oleogel formulations according to the present disclosure which have been found to display satisfactory buoyancy in gastric fluid. This list is non-exhaustive.

TABLE 2

| API | Oil base | Polymer | Hard wax |
|---|---|---|---|
| AMX | Labrafac | EC 50 4% | Suppocire AML |
| AMX | Labrafac | EC 50 4% | Suppocire AM |
| AMX | Labrafil | EC 50 4% | Suppocire AM |
| AMX | Maisine | EC 50 4% | Suppocire AM |
| AMX | Maisine | EC 50 4% | Suppocire AML |
| AMX | Peceol | EC 50 4% | Suppocire AM |
| AMX | Peceol | EC 50 4% | Suppocire AML |
| APAP¹ | Capryol | EC 50 4% | Suppocire AM |
| APAP | Capryol | EC 50 4% | Suppocire AML |
| APAP | Labrafac | EC 50 4% | Suppocire AM |
| APAP | Labrafac | EC 50 4% | Suppocire AML |
| APAP | Labrafil | EC 50 4% | Suppocire AM |
| APAP | Labrafil | EC 50 4% | Suppocire AML |
| APAP | Lauroglycol | EC 50 4% | Suppocire AM |
| APAP | Lauroglycol | EC 50 4% | Suppocire AML |
| APAP | Maisine | EC 50 4% | Suppocire AM |
| APAP | Maisine | EC 50 4% | Suppocire AML |
| APAP | Peceol | EC 50 4% | Suppocire AM |
| APAP | Peceol | EC 50 4% | Suppocire AML |
| APAP | Plurol Oleique | EC 50 4% | Suppocire AM |
| APAP | Plurol Oleique | EC 50 4% | Suppocire AML |

¹APAP = Paracetamol = acetaminophen

The present inventors have found that the rate of release of active agent from a gel composition of the present disclosure is affected by various factors, including the choice of oily base. Typically, the higher the HLB (hydrophilic-lipophilic balance) of the oily base, the faster the rate of release. Table 3 lists seven oily bases useful in compositions of the present disclosure, in order of the rate of drug release from these compositions in vitro, from fastest at the top to slowest at the bottom.

TABLE 3

| Brand | Chemical name | HLB |
|---|---|---|
| Capryol | Propyleneglycol monocaprate | 6 |
| Labrafil 1944CS | Oleoyl macrogol 6 glycerides | 4 |
| Lauroglycol | Propyleneglycol mono laurate | 4 |
| Peceol | Glycerol mono oleate | 3 |
| Maisine 35-1 | Glyceryl monolinoleate | 4 |
| Labrafac | Propyleneglycol dicaprylocaprate | 2 |
| Plurol Oleique | Polyglyceryl 3 dioleate | 3 |

The viscosity of the oleogel also affects its performance in vivo as a gastric retentive raft. Viscosity tests were carried out to investigate the impact of the type and grade of polymeric organogelator on the viscosity of oleogels according to the present disclosure. The measurements were made using a HAAKE viscosimeter spindle TR9 and TR10. The results are summarised in Table 4 below. As will be observed, an increase in polymer grade was associated with an increase in the viscosity of the oleogel; an increase in polymer concentration was also associated with an increase in the viscosity of the oleogel; the type of oily base also had an apparent effect on viscosity; and the addition of an active agent was associated with an increase in the viscosity of the oleogel.

TABLE 4

| Oil base | Polymer | Conc (%) | η (Pas) |
|---|---|---|---|
| Labrafil 1944 CS | Ethylcellulose 14 cps | 2 | 0.15 |
| Labrafil 1944 CS | Ethylcellulose 14 cps | 3 | 0.28 |
| Labrafil 1944 CS | Ethylcellulose 14 cps | 4 | 0.51 |
| Labrafil 1944 CS | Ethylcellulose 50 cps | 2 | 0.35 |
| Labrafil 1944 CS | Ethylcellulose 50 cps | 3 | 1.21 |
| Labrafil 1944 CS | Ethylcellulose 50 cps | 4 | 4.22 |
| Capryol 90 | Ethylcellulose 50 cps | 4 | 0.61 |
| Capryol 90 | Ethylcellulose 50 cps | 8 | 7.14 |
| Capryol 90 | Ethylcellulose 100 cps | 8 | 22.00 |
| Peceol | Ethylcellulose 50 cps | 4 | 8.66 |
| Labrafac | Ethylcellulose 50 cps | 4 | 1.50 |
| Lauroglycol (LG) | Ethylcellulose 50 cps | 4 | 0.61 |
| Acyclovir 100 + LG | Ethylcellulose 50 cps | 4 | 2.00 |

The type of hard wax or wax-like additive used in the formulations of the present disclosure has been found to have an impact on the rate of release of the active agent from the formulation, as summarised in Table 5 below:

TABLE 5

| | MP | MAISINE 4% EC | | PECEOL 4% EC | |
|---|---|---|---|---|---|
| | (° C.) | T50%(h) | T80%(h) | T50%(h) | T80%(h) |
| APAP | | | | | |
| Gelucire | 43 | 2 | 6.5 | 2 | Nd² |
| 43/01 | | | | | |
| Suppocire | 37 | 8 | >24 | 5 | 18 |
| AM | | | | | |
| Geleol | 57 | 4 | 10 | 4 | 12 |
| AMX | | | | | |
| Gelucire | 43 | 2 | 5 | T20% = 11 h | Nd |
| 43/01 | | | | | |
| Suppocire | 37 | 4 | Nd | T35% = 20 h | Nd |
| AM | | | | | |
| Geleol | 57 | T30% = 6 h | Nd | 120% = 20 h | Nd |

²Nd: dissolution test stopped before reaching T80%

In this table, Ta % denotes the time (in hours) taken for a % of the active agent to be released in vitro (T50%=time taken for 50% release etc.).

### EXAMPLES

The present disclosure may be further comprehended by reference to the specific examples and figures below, which are purely exemplary and are not limiting in scope.

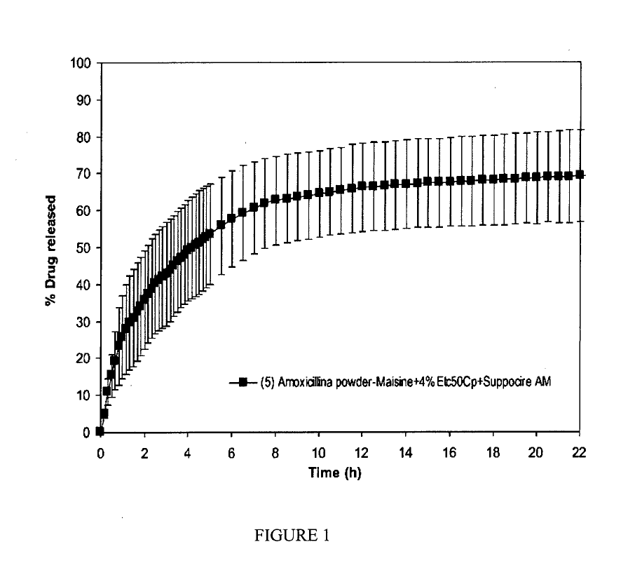

FIG. 1 illustrates the results of a dissolution test performed on the composition of example 1.

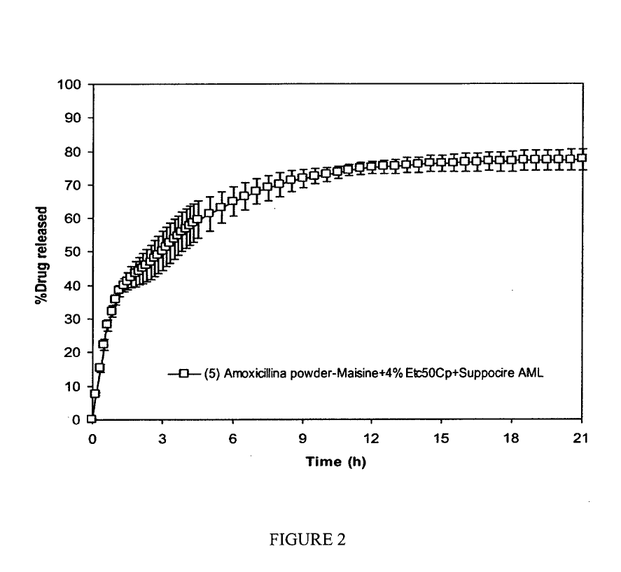

FIG. 2 illustrates the results of a dissolution test performed on the composition of example 2.

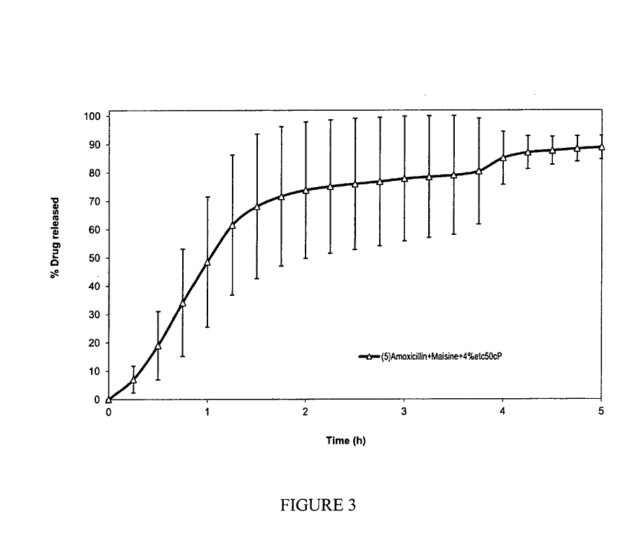

FIG. 3 illustrates the results of a dissolution test performed on the composition of comparative example 3 (no hard wax additive).

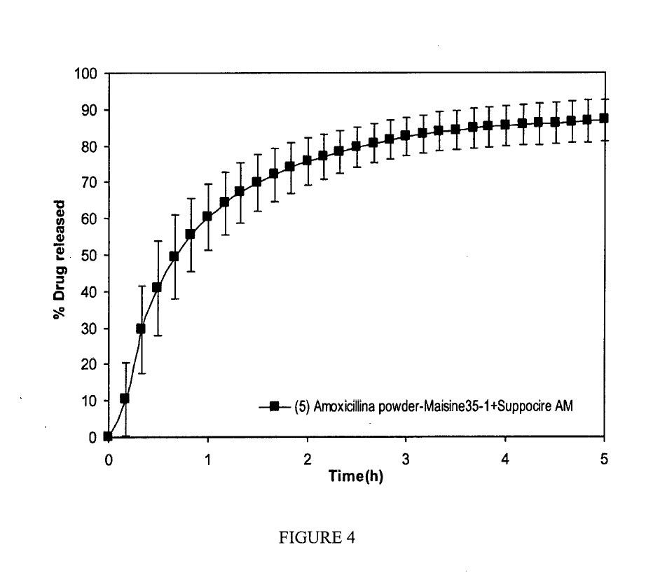

FIG. 4 illustrates the results of a dissolution test performed on the composition of comparative example 4 (no polymer).

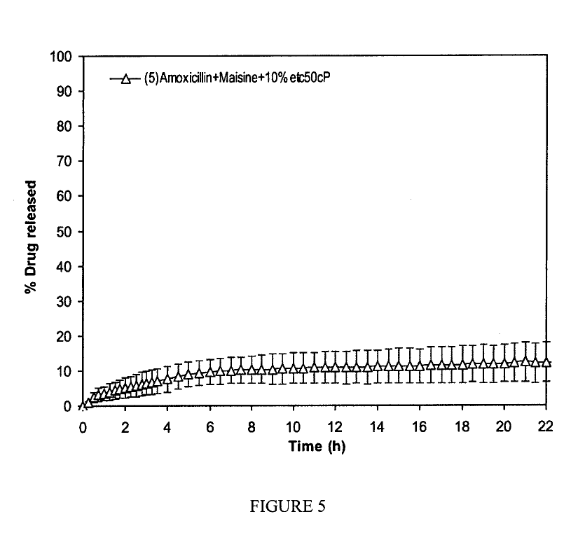

FIG. 5 illustrates the results of a dissolution test performed on the composition of comparative example 5 (no hard wax additive).

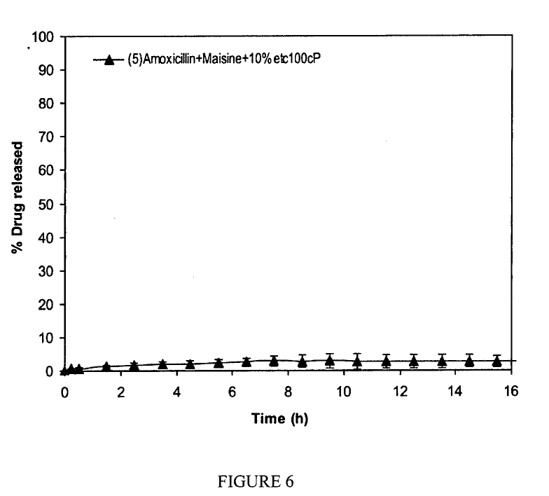

FIG. 6 illustrates the results of a dissolution test performed on the composition of comparative example 6 (no hard wax additive).

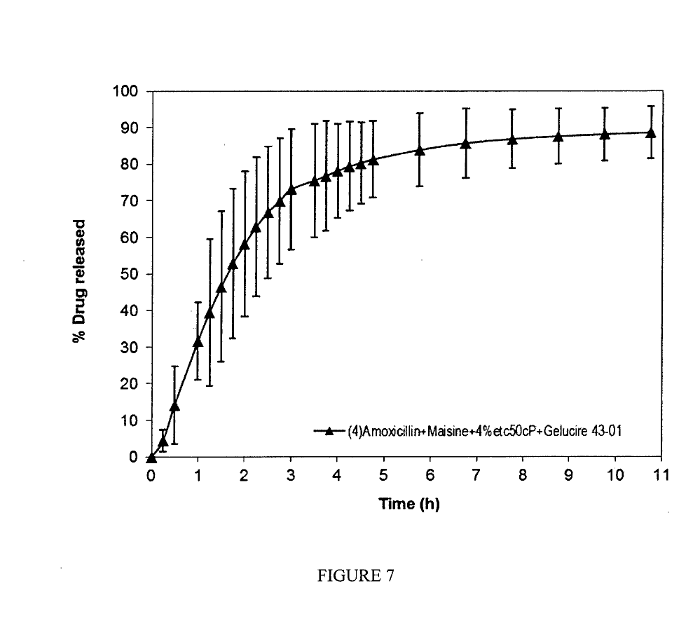

FIG. 7 illustrates the results of a dissolution test performed on the composition of example 7.

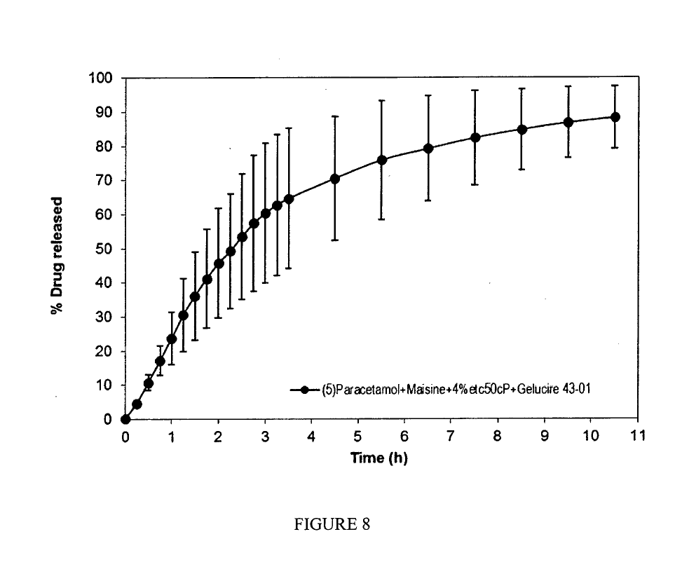

FIG. 8 illustrates the results of a dissolution test performed on the composition of example 8.

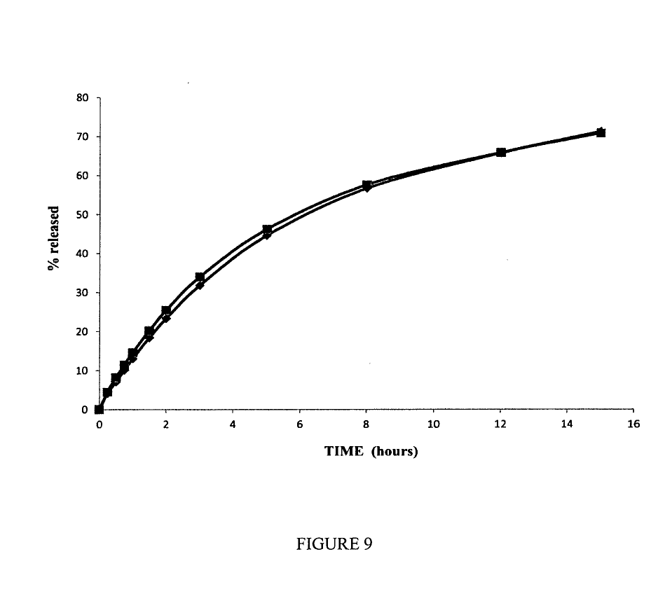

FIG. 9 shows the dissolution profile of the composition of example 12 in acetate buffer (pH 4.5) plus 0.2% sodium lauryl sulfate (experiment performed in duplicate).

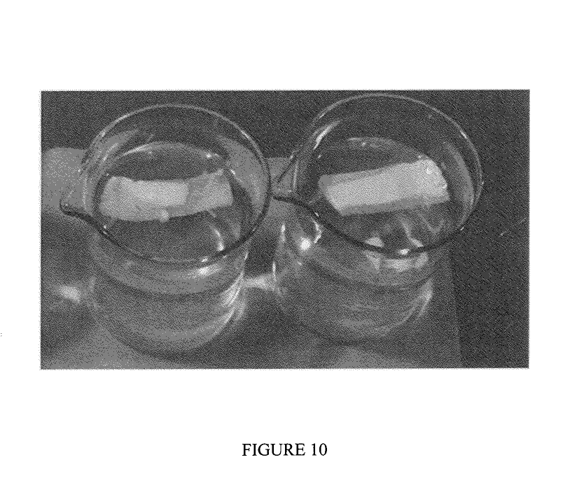

FIG. 10 illustrates a floating/buoyancy test performed on the composition of example 12 in water (experiment performed in duplicate).

### EXAMPLE 1: AMOXICILLIN POWDER+MAISINE-4% ETC50CP-SUPPOCIRE AM

Component (a): Maisine-4% Etc50Cp:

Maisine 35-1 batch 126879, Gattefosse (FR)

Ethylcellulose Type ECN 50 batch 41796, Pharm Hercules

Warm up Maisine to 90° C. whilst stirring with a magnetic stirrer. Add 4% ethylcellulose and stir until the ethylcellulose dissolves completely.

Component (c): Suppocire AM

Suppocire AM Pellets batch 2E0905-2, Gattefosse SAS (FR)

Warm up 20 g of Maisine-4% Etc50Cp and 4 g of Suppocire AM to 37° C. while stirring. The compound is elastic.

Component (b): Amoxicillin Powder

Amoxicillin trihydrate powder batch WJAN1506

Weigh 12.00 g of Maisine-4% Etc50Cp+Suppocire AM preparation and add 6.888 g of Amoxicillin trihydrate.

The resulting oleogel composition is buoyant in gastric fluid and capable of holding together as a raft in the gastric environment.

A dissolution test was performed using apparatus 2 (paddle), rotation speed 100 rpm, temperature 37° C. in 1000 mL of distilled water. The sample for the dissolution test was prepared by weighing 3.148 g of the AMX oleogel (containing Amoxicillin-Maisine-4% Etc50Cp+Suppocire AM) directly onto greaseproof paper.

The composition was found to float at the surface of the dissolution medium, forming a continuous large and thin raft around the paddle shaft. The amount of drug released was determined by UV detection at 275 nm.

The results of the dissolution test are shown in FIG. 1. As will be seen, controlled sustained drug release was attained with a T50% of 4 hours. The results showed moderate variability.

### EXAMPLE 2: AMOXICILLIN POWDER+MAISINE-4% ETC50CP-SUPPOCIRE AML

Component (a): Maisine-4% Etc50Cp:

Maisine 35-1 batch 126879, Gattefosse (FR)

Ethylcellulose Type ECN 50 batch 41796, Pharm Hercules

Warm up Maisine to 90° C. whilst stirring with a magnetic stirrer. Add 4% ethylcellulose and stir until the ethylcellulose dissolves completely.

Component (c): Suppocire AML

Suppocire AML Pellets batch 9E0303-2, Gattefosse SAS (FR)

Warm up 20 g of Maisine-4% Etc50Cp and 10 g of Suppocire AML to 37° C. while stirring. The compound is elastic.

Component (b): Amoxicillin Powder

Amoxicillin trihydrate powder batch WJAN1506

Weigh 12.00 g of Maisine-4% Etc50Cp+Suppocire AML preparation and add 6.888 g of Amoxicillin trihydrate.

A dissolution test was performed as in Example 1. The composition was found to float at the surface of the dissolution medium, forming a continuous large and thin raft around the paddle shaft.

The results of the dissolution test are shown in FIG. 2. As will be seen, controlled sustained drug release was attained with a T50% of 3 hours. The results showed low variability (small error bars) and hence good reproducibility. The small amount of lecithin contained in Suppocire AML allows faster dissolution and drug release as compared with the formulation containing Suppocire AM (Example 1).

### COMPARATIVE EXAMPLE 3: AMOXICILLIN POWDER+MAISINE-4% ETC50CP

(No hard wax additive included)

Component (a): Maisine-4% Etc50Cp:

Maisine 35-1 batch 126879, Gattefosse (FR)

Ethylcellulose Type ECN 50 batch 41796, Pharm Hercules

Warm up Maisine to 90° C. whilst stirring with a magnetic stirrer. Add 4% ethylcellulose and stir until the ethylcellulose dissolves completely.

Component (b): Amoxicillin Powder

Amoxicillin trihydrate powder batch WJAN1506

Weigh 12.00 g of Maisine-4% Etc50Cp and add 6.888 g of Amoxicillin. The compound is creamy and yellow.

A dissolution test was performed using apparatus 2 (paddle), rotation speed 100 rpm, temperature 37° C. in 1000 mL of distilled water with blank subtraction. In the dissolution medium, the formulation does not hold together as a continuous integral raft, but spreads out at the surface of the dissolution medium in multiple “small flakes” and releases its drug content very rapidly. The amount of drug released was determined by UV detection. The sample for the dissolution test was prepared by weighing 3.148 g of Amoxicillin (POWDER)-Maisine-4% Etc50Cp directly onto greaseproof paper.

The results are shown in FIG. 3. As will be seen, the drug was rapidly released from the formulation with a T50% of only 1 hour. The results were highly variable as indicated by the size of the error bars, demonstrating poor reproducibility.

### COMPARATIVE EXAMPLE 4: AMOXICILLIN POWDER+MAISINE-SUPPOCIRE AM

(No polymer included hence this is not an oleogel formulation)

Maisine 35-1

Suppocire AM, Gattefosse (FR)

Amoxicillin trihydrate powder batch WJAN1506

Warm up 5 g of Suppocire AM at temperature>37° C. and add while stirring 18 g of Maisine. Weigh 12.00 g of the resulting composition and add 6.888 g of Amoxicillin trihydrate.

A dissolution test was carried out according to the previous examples. In the dissolution medium, the formulation does not hold together as a continuous integral raft, but breaks apart, spreads out and floats in multiple small flakes, releasing its drug content very rapidly.

The results of the dissolution test are shown in FIG. 4. As will be seen, the drug was rapidly released from the formulation with a T50% of less than 1 hour.

### COMPARATIVE EXAMPLE 5: AMOXICILLIN POWDER+MAISINE-10% ETC50CP

(No hard wax additive included)

Component (a): Maisine-10% Etc50Cp:

Maisine 35-1 batch 126879, Gattefosse (FR)

Ethylcellulose Type ECN 50 batch 41796, Pharm Hercules

Warm up Maisine to 90° C. whilst stirring with a magnetic stirrer. Add 10% ethylcellulose and stir until the ethylcellulose dissolves completely.

Component (b): Amoxicillin Powder

Amoxicillin trihydrate powder batch WJAN1506

Weigh 12.00 g of Maisine-10% Etc50Cp and add 6.888 g of Amoxicillin.

A dissolution test was performed using apparatus 2 (paddle), rotation speed 100 rpm, temperature 37° C. in 1000 mL of distilled water with blank subtraction. The amount of drug released was determined by UV detection. The sample for the dissolution test was prepared by weighing 3.148 g of Amoxicillin (POWDER)-Maisine-10% Etc50Cp directly onto greaseproof paper.

The formulation does not spread out at the surface of the dissolution medium but sinks to the bottom of the vessel. The resulting dissolution rate is extremely slow with drug strongly entrapped in the agglomerated formulation. The results of the dissolution test are shown in FIG. 5.

### COMPARATIVE EXAMPLE 6: AMOXICILLIN POWDER+MAISINE-10% ETC100CP

(No hard wax additive included)

Component (a): Maisine-10% Etc100Cp: Maisine 35-1 batch 126879, Gattefosse (FR) Ethylcellulose Type ECN 100 batch 42324, Pharm Hercules

Warm up Maisine to 90° C. whilst stirring with a magnetic stirrer. Add 10% ethylcellulose and stir until the ethylcellulose dissolves completely.

Component (b): Amoxicillin Powder

Amoxicillin trihydrate powder batch WJAN1506

Weigh 12.00 g of Maisine-10% Etc100Cp and add 6.888 g of Amoxicillin.

A dissolution test was performed using apparatus 2 (paddle), rotation speed 100 rpm, temperature 37° C. in 1000 mL of distilled water with blank subtraction. The amount of drug released was determined by UV detection. The sample for the dissolution test was prepared by weighing 3.148 g of Amoxicillin (POWDER)-Maisine-10% Etc100Cp directly onto greaseproof paper.

The formulation does not spread out at the surface of the dissolution medium but sinks to the bottom of the vessel. The resulting dissolution rate is extremely slow with drug strongly entrapped in the agglomerated formulation. The results of the dissolution test are shown in FIG. 6.

### EXAMPLE 7: AMOXICILLIN POWDER-MAISINE-4% ETC 50CP+GELUCIRE 43/01

Component (a): Maisine-4% Etc50Cp:

Maisine 35-1 batch 126879, Gattefosse (FR)

Ethylcellulose Type ECN 50 batch 41796, Pharm Hercules

Warm up Maisine to 90° C. whilst stirring with a magnetic stirrer. Add 4% ethylcellulose and stir until the ethylcellulose dissolves completely.

Component (c): Gelucire 43/01

Gelucire 43/01 batch 1E5203-2-2, Gattefosse SAS (FR)

Warm up 12.5 g of Maisine-4% Etc50Cp and 3.5 g of Gelucire 43/01 to 45° C. while stirring. The compound is elastic.

Component (b): Amoxicillin Powder

Amoxicillin trihydrate powder batch WJAN1506

Weigh 12.00 g of Maisine-4% Etc50Cp+Gelucire 43/01 preparation and add 6.888 g of Amoxicillin trihydrate.

A dissolution test was performed as in Example 1. The composition floats at the surface of the dissolution medium forming a continuous large and thin raft around the paddle shaft.

The results of the dissolution test are shown in FIG. 7. As will be seen, controlled sustained drug release was attained with a T50% of 2 hours and a T80% at 5 hours.

### EXAMPLE 8: APAP (PARACETAMOL) MAISINE-4% ETC 50-GELUCIRE 43/01

Component (a): Maisine-4% Etc50Cp:

Maisine 35-1 batch 126879, Gattefosse (FR)

Ethylcellulose Type ECN 50 batch 41796, Pharm Hercules

Warm up Maisine to 90° C. whilst stirring with a magnetic stirrer. Add 4% ethylcellulose and stir until the ethylcellulose dissolves completely.

Component (c): Gelucire 43/01

Gelucire 43/01 batch 1E5203-2, Gattefosse SAS (FR)

Warm up 12.5 g of Maisine-4% Etc50Cp and 3.5 g of Gelucire 43/01 to 45° C. while stirring. The compound is elastic.

Component (b): Paracetamol Powder

Paracetamol powder batch 6088902V768

Weigh 12.00 g of Maisine-4% Etc50Cp+Gelucire 43/01 preparation and add 2.4 g of APAP (paracetamol).

A dissolution test was performed as in Example 1. The composition floats at the surface of the dissolution medium forming a continuous large and thin raft around the paddle shaft. The results of the dissolution test are shown in FIG. 8. As will be seen, controlled sustained drug release was attained with a T50% of 2 hours. T80% is achieved within 6.5 hours.

The formulations of examples 1-8 are summarized in Table 6. As will be seen, the oleogel formulations comprising a hard wax additive (Suppocire AM, Suppocire AML or Gelucire 43/01) showed significantly improved sustained release dissolution characteristics as compared with the non-oleogel formulations and the oleogel formulations without a hard wax additive. The buoyancy and integrity of the formulations was also improved.

TABLE 6

| % | Example 1 | Example 2 | Example 3 | Example 4 | Example 5 | Example 6 | Example 7 | Example 8 |
|---|---|---|---|---|---|---|---|---|
| AMX | 36.47 | 36.47 | 36.47 | 36.47 | 36.47 | 36.47 | 36.47 | |
| APAP | | | | | | | | 16.67 |
| Maisine | 50.83 | 40.66 | 60.99 | 49.72 | 57.75 | 57.75 | 47.64 | 62.49 |
| Ethylcellulose 50 cp | 2.12 | 1.69 | 2.54 | | 6.35 | | 1.99 | 2.61 |
| Ethylcellulose 100 cp | | | | | | 6.35 | | |
| Suppocire AM | 10.59 | | | 13.81 | | | | |
| Suppocire AML | | 21.15 | | | | | | |
| Gelucire 43/01 | | | | | | | 13.90 | 18.23 |
| T 50% (h) | 4 | 3 | 1 | 40 min | Nd | Nd | 2 | 2 |
| | FIG. 1 | FIG. 2 | FIG. 3 | FIG. 4 | FIG. 5 | FIG. 6 | FIG. 7 | FIG. 8 |
| Buoyancy | Floats | Floats | Floats | Floats | Does not float | Does not float | Floats | Floats |
| in dissolution medium | | | | | | | | |
| Consistency in | Forms a raft | Forms a raft | Breaks into | Breaks into | Agglomerates | Agglomerates | Forms a | Forms a |
| dissolution medium | | | flakes | flakes | and sinks | and sinks | raft | raft |

### EXAMPLES 9 TO 13: PRAZIQUANTEL-LABRAFAC-ETC 50-GELUCIRE 43/01

Examples 9, 12 and 13 illustrate the present invention, whilst Examples 10 and 11 are comparative, lacking a hard wax additive and an organogelator respectively. These examples all utilize Praziquantel as active ingredient. Details of Examples 9-12 are summarized in Table 7.

TABLE 7

| | | | Ex11 | |
|---|---|---|---|---|
| | | Ex10 | No | |
| Ingredients (%) | Ex 9 | No hard wax | organogelator | Ex12 |
| Praziquantel | 32.95 | 38.80 | 33.61 | 33.27 |
| Labrafac PG | 49.96 | 58.84 | 50.98 | 50.46 |
| Aqualon N50 (Etc 50) | 2.00 | 2.36 | — | 1.02 |
| Gelucire 43/01 | 15.00 | — | 15.41 | 15.25 |
| Buoyancy water | OK | NO | NO | OK |
| Buoyancy buffer | OK | NO | NO | OK |

Flavouring agents and sweeteners can be added to the formulation to make it more palatable. Example 13 provides a formulation containing Praziquantel that has been flavoured to hide the bitter taste of this active ingredient. The formulation of this composition is summarised in Table 8, and the manufacturing process is described in Table 9.

TABLE 8

| | | | | Quantity |
|---|---|---|---|---|
| Ingredients Ex 13 | | | % | per unit |
| Trade name | Chemical name | Function | w/w | (g) |
| Praziquantel | Praziquantel | Active ingredient | 25.86 | 1.200 |
| EP | | | | |
| Labrafac PG | Propylene glycol | Oil phase | 53.87 | 2.500 |
| | dicaprylocaprate | | | |
| Aqualon N50 | Ethylcellulose | Organogelator | 1.94 | 0.090 |
| | 50 cP | | | |
| Gelucire 43/01 | Hard fat | Hardening agent | 14.01 | 0.650 |
| Orange | | Flavouring agent | 0.38 | 0.018 |
| Grapefruit | | | | |
| Mint | | Flavouring agent | 0.06 | 0.003 |
| Masking agent | | Taste masking | 0.63 | 0.029 |
| Sucralose | | Sweetener | 3.25 | 0.151 |
| Total per unit | | | | 4.641 |
| (stick pack) | | | | |

A dissolution test was performed on the composition of example 12 using USP Apparatus 2 (paddle), rotation speed 100 rpm, temperature 37° C. in 900 mL of acetate buffer (pH 4.5) plus 0.2% sodium lauryl sulfate. The amount of drug released was determined by UV detection. The system is extracted from the stick pack and dropped in the dissolution vessel for testing. The system is floating at the surface of the dissolution fluid for at least the duration of the test. The dissolution profile of the composition of example 12 is illustrated in FIG. 9. Its buoyancy in distilled water after stick pack extraction is illustrated in FIG. 10.

### EXAMPLE 14: ALFUZOSIN PELLETS FOR OLEOGEL PREPARATION

The active agent in the compositions of the present disclosure may be included in the form of a powder, as in the examples above. Other formulations are however possible, including sustained or delayed release formulations. A pellet formulation for alfuzosin is prepared as below:

### Alfuzosine Pellet Centesimal Formula

| | % |
|---|---|
| Core | |
| Alfuzosin HCl | 3.50 |
| Compritol 888ATO - glyceryl behenate | 10.51 |
| Avicel -microcrystalline cellulose | 3.50 |
| PVP plasdone K29-32 | 1.63 |
| Mounting blend | |
| Methocel K100M - HPMC | 20.77 |
| PVP plasdone K29-32 | 2.79 |
| Compritol 888 ATO -glyceryl behenate | 20.77 |
| Coatings | |
| 1- Opadry YS -HPMC 3 cps | 6.35 |
| 2- Surelease - EC | 21.08 |
| 3- Opadry YS -HPMC 3 cps | 9.09 |
| | 100.00 |

The components are introduced into the bowl of a fluid bed rotogranulator, then initial core formation is obtained by spraying water while agglomerates are formed by the twisting plate.

Then ingredients for mounting are added to the cores and agglomeration is obtained again by spraying water while agglomerates are shaped by the twisting plate. 3 successive coating layers are applied to the pellets thus obtained, a first one based on HPMC 3 cps (weight gain of 10%), the second one based on Ethylcellulose polymer (weight gain of 30%) and finally another HPMC based layer (weight gain of 10%).

The finished pellets are then ready to be added to an oleogel base formulation according to the present disclosure.

1. A gastric retentive gel composition comprising:
(a) a hydrophobic or amphiphilic liquid gelled with an organogelator;
(b) an active agent; and
(c) a hard wax or wax-like additive.

2. The gel composition of claim 1, wherein (a) comprises an organogel, lipogel or oleogel, and (b) and (c) are dispersed, solubilized or suspended in the gel.

3. The gel composition of claim 1, having a density less than the density of gastric fluid (approximately 1.004-1.1 g/ml).

4. The gel composition of claim 1, wherein the hard wax or wax-like additive is selected from triglycerides, mono- and diglycerides, natural or synthetic waxes, fat or wax alcohols, fatty acids, esters of fatty alcohols and fatty acids, fatty acid amides, or hardened fats or oils.

5. The gel composition of claim 1, wherein the hard wax or wax-like additive is selected from a semi-synthetic glyceride base comprising saturated C8-18 triglyceride fatty acids; a mixture of triglycerides, diglycerides and monoglycerides; a mixture of glycerides and esters of polyethylene glycol; mono- and diglycerides, glycerol monostearate; hydrogenated castor oil; a glyceryl stearate; hydrogenated palm oil; triglyceride of behenic acid; glyceryl behenate behenoyl polyoxyl-8 glycerides); glyceryl distearate; a glyceryl stearate; 114—trimyristate; 116—tripalmitate; 118—tristearate, and equivalents of any of the foregoing.

6. The gel composition of claim 1, wherein (c) is provided in a quantity between about 5-200% by weight of (a).

7. The gel composition of claim 1, wherein the organogelator comprises an ethylcellulose polymer, preferably an ethylcellulose polymer having a viscosity grade of about 50 centipoise (cP) or less; or wherein the organogelator comprises polyethylene, polyethyleneoxide propyleneoxide, gum such as xanthan gum, guar gum or locust bean gum, polyacrylic acid or polyacrylic-methacrylic acid such as ammonio methacrylate copolymer, shellac resin, polyvinyl acid, carboxyvinyl polymer such as carbomers, or block copolymer ethyleneoxide-co-propyleneoxide including poloxamers.

8. The gel composition of claim 1, wherein the hydrophobic or amphiphilic liquid comprises an oil or a modified oil.

9. The gel composition of claim 1, wherein the hydrophobic or amphiphilic liquid comprises a glyceride such as a mono-, di- or tri-glyceride, or wherein the hydrophobic or amphiphilic liquid is selected from propyleneglycol monocaprate, propyleneglycol dicaprylocaprate, oleoyl macrogol 6 glycerides, propyleneglycol monolaurate, glyceryl monolinoleate, glycerol mono oleate, polyglyceryl 3 dioleate, a medium chain triglyceride, and equivalents thereof.

10. The gel composition of claim 1, wherein a weight/weight (w/w) ratio of organogelator to hydrophobic or amphiphilic liquid in component (a) is up to about 15:85 (15% organogelator).

11. The gel composition of claim 1, wherein the w/w ratio of organogelator to hydrophobic or amphiphilic liquid in (a) is between about 1:99-5:95 (1-5% organogelator), preferably between about 3:97-5:95 (3-5% organogelator); or is about 4:96 (4% organogelator) or less.

12. The gel composition of claim 1, wherein (a) comprises a hydrophobic or amphiphilic liquid gelled with an ethylcellulose polymer with a viscosity grade of about 50 cP, and the w/w ratio of the ethylcellulose polymer to the hydrophobic or amphiphilic liquid is about 4:96 (4% ethylcellulose polymer).

13. The gel composition of claim 1, wherein the active agent comprises a pharmaceutical drug, a nutritional supplement, a marker such as a diagnostic marker, or an imaging agent, or wherein the active agent is selected from active pharmaceuticals, prodrugs of active pharmaceuticals, active biologicals, antibiotics, antifungals, antitoxins, antigens, therapeutics, preventive therapeutics, analgesics, nutritional supplements, imaging agents, fluid stabilizers, or any combination thereof.

14. The gel composition of claim 1, wherein the active agent is suitable for administration to or through the upper part of the gastrointestinal tract, in particular to or through the stomach.

15. The gel composition of claim 1 wherein the active agent is an analgesic such as paracetamol; or an antibiotic such as amoxicillin; or a receptor agonist or antagonist such as alfuzosin HCl, atenolol or losartan; or an anti-viral drug such as acyclovir; or a beta blocker such as propranolol.

16. The gel composition of claim 1, wherein the active agent comprises up to about 50% by weight of the gel composition.

17. The gel composition of claim 1, wherein the active agent comprises at least about 25% by weight of the gel composition.

18. The gel composition of claim 1, wherein the active agent is formulated as a powder, as pellets including coated pellets, as particles, nanoparticles or granules, or as a liquid or gel; or wherein the active agent is formulated for controlled, sustained or modified release such as capsules, pellets, non pareils, microballoons, microspheres or beads.

19. The gel composition of claim 1, wherein (a) comprises ethylcellulose 50cPs gelled with propyleneglycol dicaprylocaprate, glycerol mono oleate or glyceryl monolinoleate in a w/w ratio of about 4:96 (4% w/w organogelator), and (c) comprises mono- and diglycerides, glycerol monostearate, a mixture of glycerides and esters of polyethylene glycol, a semi-synthetic glyceride base comprising saturated C8-18 triglyceride fatty acids.

20. The gel composition of claim 1, wherein (b) comprises paracetamol, amoxicillin or alfusozine.

21. The gel composition of claim 1, comprising:
15-90% w/w, or preferably 30-80% w/w, hydrophobic or amphiphilic liquid;
0.2-15% w/w, or preferably 1-4% w/w, organogelator;
0.1-50% w/w, or preferably 10-50% w/w, active agent; and
2-70% preferably 10-60% w/w, hard wax or wax-like additive;
wherein the sum of these percentages is 100%.

22. (canceled)

23. A dosage form which is a sachet, bottle, tube, dosing syringe, or a bottle with a dosing pump, comprising the composition of claim 1.

24. A dosage form which is a single dose sachet containing a predetermined quantity of the composition of claim 1 or a bottle, tube or syringe that is configured to dispense a predetermined quantity of the composition of claim 1, wherein the predetermined quantity represents a dose of the active agent.

25. A method for administering an active agent to or through the upper gastrointestinal tract, in particular the stomach, of a human or animal patient, comprising orally administering to the patient a gel composition according to claim 1 which comprises said active agent.

26. (canceled)

27. (canceled)

28. (canceled)

29. The method of claim 25, wherein the patient is in a non-fasting state.

30. (canceled)
